(19) United States
(12) Patent Application Publication
(10) Pub. No.: US 2008/0118589 A1
(43) Pub. Date: May 22, 2008

(54) Pharmaceutical formulations of rhodiola crenulata and methods of use thereof

(76) Inventors: Sallie Smith Schneider, Longmeadow, MA (US); Kalidas Shetty, Amherst, MA (US)

Correspondence Address: BROMBERG & SUNSTEIN LLP, 125 SUMMER STREET, BOSTON, MA, 02110-1618, US

(21) Appl. No.: 11/432,772

(22) Filed: May 11, 2006

Related U.S. Application Data
(60) Provisional application No. 60/680,106, filed on May 11, 2005.

(51) Int. Cl. A61K 36/00; A61P 43/00

(52) U.S. Cl. 424/773

(57) ABSTRACT

Isolated extracts of Rhodiola crenulata and their constituents sensitize tumor cells to cancer therapies, such as tamoxifen, retinoids, and radiation, and act to potentiate radiation therapy in the treatment of cancer, with or without adjuvant chemotherapy. Methods are provided for inhibiting the P13kinase/AKT pathway in cancer cells, a general survival pathway which leads to resistance of many tumors to typical therapies; decreasing cyclooxygenase (COX) activities in cancer cells, another pathway known to increase resistance to tumors; inhibiting telomerase activity in cancer cells, an activity notable for its ability to increase the life span of the cells; enhancing the ATM (Ataxia-Telangiectasia Mutated) response in cells, which is involved in DNA damage sensing and repair; modulating cellular response to oxidation, which effects the other signaling pathways on numerous levels; and altering usage of metabolic energy pathways.

### REFERENCE TO RELATED APPLICATIONS

This application claims priority from U.S. Provisional Application Ser. No. 60/680,106 filed May 11, 2005, the entire contents of which are hereby incorporated by reference herein.

### GOVERNMENT FUNDING

This invention was made in part with government support under grant DE-FG-02-03ER63670, awarded by the United States Department of Energy. The government has certain rights in the invention.

### TECHNICAL FIELD

The present invention relates to isolated natural products from the Rhodiola crenulata species, and methods for potentiating chemotherapy and/or radiation therapy, and other uses thereof.

### STATEMENT OF COOPERATIVE RESEARCH AGREEMENT

The present invention, as defined by the claims herein, was made by parties to a Joint Research Agreement (“Agreement”) between the University of Massachusetts, Amherst and Baystate Medical Center, Inc., as a result of activities undertaken within the scope of that Agreement. The Agreement was in effect prior to the date of the invention.

### BACKGROUND ART

Rhodiola is a plant that grows in the high altitude regions of Siberia and Tibet. There are numerous species of the genus Rhodiola, the most well studied being Rhodiola rosea. Usage in Chinese and Tibetan medicine has been documented for centuries. In general, the root was and is used for treating depression, fatigue, and stress. However, it has also been used to treat sexual and reproductive problems, altitude sickness and disorders of the nervous system (reviewed in Brown et al., (2002) Rhodiola rosea: a phytomedicinal review Herbalgram 56:40-52). More recent research has suggested that it can be used to enhance insulin in diabetic rats (Molokovskii et al., 1989, Probl Endokrinol 35(6):82-87), prevent hypoxic injury of the pancreas, increase amylase activity (Kobayashi et al., 2003, Biol Pharm Bull 26(7):1045-1048), enhance ATP content in skeletal muscle (Abidov et al., 2003, Bull Exp Biol Med 136(6):585-587), protect from cardiac damage (Maslova et al., 1994, Eksp Klin Farmakol 57(6):61-63; Lishmanov et al., 1997, Eksp Klin Farmakol. 60(3):34-36) and to prevent mutagenensis (Salikhova et al., 1997, Patol Fiziol Exsp Ter 4:22-24; Duhan et al., 1999,) Tsitol Genet. 33(6):19-25.).

Extracts and compounds from other Rhodiola species, in particular Rhodiola rosea, seem to have possible benefits as anti-cancer agents (e.g. Razina et al., 2000, Eksp Klin Farmakol, 63(5):59-61 For example, Russian studies indicate that Rhodiola extracts decrease the growth of a lung cancer graft and an ascites tumor in rodents. Specifically, the chemotherapeutic agents cyclophosphamide, rubomycin, and adriamycin, appear to work with Rhodiola to decrease the growth and prevent metastases, and Rhodiola appears to be able to prevent the toxic effects from certain therapeutic agents on the liver and on blood generation (see Udinstev et al., 1991, Eur J Cancer 27(9): 1182; and Udinstev et al., 1992, Vopr Onol 38(10):1217-1222). Anecdotal evidence discussed on the internet suggests that Rhodiola rosea is radioprotective (see, for example, AMERIDEN® International, Ameri-News from Dr. Howard Peiper & Richard L. Hall, CEO, October, 2003 [online], [retrieved on Apr. 27, 2005]. Retrieved from the Internet URL:http:/www.ameriden.com/newsletter_sample.htm).

To date, there are no studies reporting such benefits from treatment with extracts from the species Rhodiola crenulata, and no reports of studies performed showing the effects of Rhodiola crenulata extracts or components, either alone or in concert with other agents or treatments on breast cancer, colon cancer, melanoma, leukemia, and other human cancers.

U.S. Pat. No. 6,399,116, to R. Xui (the '116 patent) is directed to Rhodiola, particularly Rhodiola crenulata, for the treatment of various conditions, and especially to methods of “enhancing blood oxygen in a subject having muscle fatigue,” raising skeletal muscle levels of ATP, and/or reducing blood levels of lactate. Xui states that Rhodiola extracts can be administered to a subject to enhance blood oxygen levels, enhance working capacity and endurance, to enhance memory and concentration, to enhance cardiac and cardiovascular function, to provide antioxidant effects, to protect against oxidation, to modulate testosterone and estradiol levels, to modulate sleep, to improve sexual ability, such as improve sexual performance, and to treat other conditions and diseases.

The '116 patent also discloses generally that extracts and compounds from Rhodiola, preferably Rhodiola crenulata, have other useful and beneficial effects, including “to provide anti-cancer effects, to promote DNA repair, to provide anti-radiation effects, to protect against radiation” and many others, but these claims appear to be anecdotal as none are supported by explanations, illustrated by examples, research protocols, or data, and none include citations of previous studies by others.

### SUMMARY OF THE INVENTION

The invention disclosed and claimed herein provides embodiments wherein isolated extracts of Rhodiola crenulata and their constituents sensitize tumor cells to cancer therapies, such as tamoxifen, retinoids, and radiation. Other embodiments provide methods for potentiating radiation therapy in the treatment of cancer, with or without adjuvant chemotherapy. Other embodiments in accordance with the invention provide methods for enhancing the effects of hormone modulators, such as tamoxifen and natural and synthetic retinoids, (either synergistically or additively), methods for reducing tumor cell growth and enhancing death (i.e., increasing necrosis in cancer cells) by administering therapeutically effective amounts of isolated extract products from Rhodiola crenulata. Specifically, embodiments in accordance with the present invention provide methods for sensitizing cancer cells by administering effective doses of isolated Rhodiola crenulata extracts.

Particular embodiments provide methods for inhibiting the P13kinase/AKT pathway in cancer cells, a general survival pathway which leads to resistance of many tumors to typical therapies, thus also decreasing AKT phosphorylation in cancer cells; decreasing cyclooxygenase (COX) activities in cancer cells, another pathway known to increase resistance of tumors; inhibiting telomerase activity in cancer cells, an activity notable for its ability to increase the life span of the cells; enhancing the ATM (Ataxia-Telangiectasia Mutated) response in cells, which is involved in DNA damage sensing and repair; modulating cellular response to oxidation, which effects the other signaling pathways on numerous levels; and altering usage of metabolic energy pathways.

In one particular embodiment there is provided a method for potentiating chemotherapy in a subject being treated with chemotherapy, the method comprising providing an isolated Rhodiola crenulata extract product comprising a Rhodiola organic extract, aqueous extract, acid extract, neutral extract, essential oil, isolated compound or chemically synthesized equivalent thereof and administering an effective amount of the isolated Rhodiola crenulata product to the subject being treated with chemotherapy, wherein chemotherapy treatment in the subject is potentiated relative to such treatment in the absence of the Rhodiola extract product.

Related embodiments provide a method for potentiating chemotherapy in a subject wherein the subject is being treated with at least one of tamoxifen, paclitaxel, docetaxel, epothilone, a retinoid, progesterone, cyclophospharnide, methotrexate, bleomycin, cisplatin, oxaliplatin, carboplatin, nitrogen mustards, melphalan, mechlorethamine, dacarbazine, lomustine, carmustine, chlorambucil, vinca alkaloids including vincristine and vinblastine, topotecan, ironotecan, etoposide, doxorubicin, idarubicin, epirubicin, daunorubicin, mitoxantrone and related molecules, gemcitabine, 5-fluorouracil, mitomycin C, any functional equivalent thereof, or any combination thereof. In more particular related embodiments, a method for potentiating chemotherapy in a subject is provided wherein the subject also receives radiation therapy.

Another particular embodiment provides a method for potentiating chemotherapy in a subject being treated with chemotherapy and radiation therapy, the method comprising providing an isolated Rhodiola crenulata extract product comprising a Rhodiola organic extract, aqueous extract, acid extract, neutral extract, essential oil, isolated compound or chemically synthesized equivalent thereof, and administering an effective amount of the isolated Rhodiola crenulata product to the subject before, after, or concomitant with being treated with chemotherapy and radiation therapy, wherein the subject is being treated with at least one of tamoxifen, paclitaxel, docetaxel, epothilone, a retinoid, progesterone, cyclophosphamide, methotrexate, bleomycin, cisplatin, oxaliplatin, carboplatin, nitrogen mustards, melphalan, mechlorethamine, dacarbazine, lomustine, carmustine, chlorambucil, vinca alkaloids including vincristine and vinblastine, topotecan, ironotecan, etoposide, doxorubicin, idarubicin, epirubicin, daunorubicin, mitoxantrone and related molecules, gemcitabine, 5-fluorouracil, mitomycin C, any functional equivalent thereof, or any combination thereof, wherein chemotherapy and radiation therapy treatment in the subject is potentiated relative to such treatment in the absence of the Rhodiola extract product.

Related embodiments include a method for potentiating chemotherapy in a subject according to embodiments described above, wherein the subject is being treated with a chemotherapy agent comprising tamoxifen, paclitaxel, docetaxel, epothilone, a functional equivalent thereof, an analog thereof or a salt thereof. In more particular embodiments, the chemotherapy agent may be a retinoid or may comprise tamoxifen, a tamoxifen analog, a functional equivalent of tamoxifen, or a salt thereof.

Still another embodiment provides a method for promoting cell death in cancer cells the method comprising administering an isolated extract product of Rhodiola crenulata to the cancer cells such that the cancer cells show increased cell death relative to cancer cells in the absence of the isolated extract product of Rhodiola crenulata. Another embodiment provides a method for enhancing the growth arrest effect of a retinoid on cancer cells, the method comprising administering an isolated extract product of Rhodiola crenulata to the cancer cells in the presence of a retinoid such that the cancer cells show enhanced growth arrest relative to cancer cells in the absence of the isolated extract product of Rhodiola crenulata. Still another embodiment provides a method for inhibiting the progression of metastases in cancer cells, the method comprising administering an isolated extract product of Rhodiola crenulata to the cancer cells such that the cancer cells show inhibited progression of metastases relative to cancer cells in the absence of the isolated extract product of Rhodiola crenulata.

Another embodiment provides a method for decreasing telomerase activity in cancer cells, the method comprising administering an effective dose of an isolated extract product of Rhodiola crenulata to the cancer cells such that the cancer cells show decreased telomerase activity relative to cancer cells in the absence of the isolated extract product of Rhodiola crenulata. And a related embodiment provides a method for decreasing telomerase activity in cancer cells further comprising administering an effective dose of tamoxifen to the cells, wherein the cancer cells are ER-negative breast cancer cells.

One particular embodiment provides a method for decreasing cyclooxygenase activity in cancer cells, the method comprising administering an effective dose of an isolated extract product of Rhodiola crenulata to the cancer cells such that the cancer cells show decreased cyclooxygenase activity relative to cancer cells in the absence of the isolated extract product of Rhodiola crenulata, and another particular embodiment provides a method for modulating the activity of p53 in cancer cells, the method comprising administering an effective dose of an isolated extract product of Rhodiola crenulata to the cancer cells such that the cancer cells show modulated p53 activity relative to cancer cells in the absence of the isolated extract product of Rhodiola crenulata.

Still another particular embodiment provides a method for increasing ataxia telangiectasia mutated (ATM) activity in cancer cells, the method comprising administering an effective dose of an isolated extract product of Rhodiola crenulata to the cancer cells such that the cancer cells show increased ATM activity relative to cancer cells in the absence of the isolated extract product of Rhodiola crenulata.

Another particular embodiment provides a method for inhibiting utilization of the proline-linked pentose phosphate pathway in cancer cells, as indicated by at least one of an accumulation of proline, a decreased level of succinate dehydrogenase, or a decreased level of proline dehydrogenase, the method comprising administering an effective dose of an isolated extract product of Rhodiola crenulata to the cancer cells such that the utilization of the proline-linked pentose phosphate pathway in cancer cells is inhibited relative to utilization of said pathway in cancer cells in the absence of administration of the isolated extract product of Rhodiola crenulata.

Yet another particular embodiment provides a method for effecting cell death of cancer cells in a subject with cancer, the method comprising administering an effective dose of an isolated extract product of Rhodiola crenulata to the subject such that cell death of the cancer cells is effected relative to cell death of the cancer cells in the absence of administration of the isolated extract product of Rhodiola crenulata. And yet another embodiment provides a method for modulating AKT phosphorylation activity in estrogen receptor-positive or estrogen receptor-negative breast cancer cells, the method comprising administering an isolated extract product of Rhodiola crenulata to the ER-positive or ER-negative breast cancer cells such that the breast cancer cells show modulated AKT phosphorylation activity relative to breast cancer cells in the absence of the isolated extract product of Rhodiola crenulata.

Related embodiments described above provide a method wherein the cancer cells are breast, colon, ovarian, liver, head and neck, lung, pancreas, leukemia, melanoma, and uterine cancer cells or any combination thereof.

Another particular embodiment provides a pharmaceutical formulation for potentiating chemotherapy in a subject, the formulation comprising a therapeutically effective amount of an isolated Rhodiola extract product isolated from the species Rhodiola crenulata or a chemically synthesized equivalent thereof, a therapeutically effective amount of a chemotherapeutic agent, and a carrier. In related embodiments, the isolated Rhodiola extract product comprises an organic extract, aqueous extract, miscible aqueous/organic mixture extract, acid extract, base extract, neutral extract, an essential oil, or a combination thereof. Particularly, the isolated extract product comprises an aqueous extract. More particularly the isolated extract product comprises an organic extract. Still more particularly the isolated extract product comprises a neutral extract. In another related particular embodiment, the isolated extract product comprises a miscible aqueous/organic mixture extract. In yet another related particular embodiment, the isolated extract product comprises an aqueous methanol extract. And in still another related particular embodiment the isolated extract product comprises an essential oil.

One particular embodiment provides a pharmaceutical formulation for potentiating chemotherapy in a subject according as described above, wherein the chemotherapy agent comprises tamoxifen, paclitaxel, docetaxel and related molecules, epothilone, a retinoid, progesterone, cyclophosphamide, methotrexate, bleomycin, cisplatin, oxaliplatin, carboplatin, nitrogen mustards, melphalan, mechlorethamine, dacarbazine, lomustine, carmustine, chlorambucil, vinca alkaloids including vincristine and vinblastine, topotecan, ironotecan, etoposide, doxorubicin, idarubicin, epirubicin, daunorubicin, mitoxantrone and related molecules, gemcitabine, 5-fluorouracil, mitomycin C, any functional equivalent thereof, or any combination thereof. A related embodiment provides a pharmaceutical formulation for potentiating chemotherapy in a subject, wherein the Rhodiola product is an isolated extract that comprises a flavonoid, a condensed flavonoid, a cyanoglycoside, a salidroside, tyrosol, a sterol, a sachaliside, a flavonoid glycoside, a cinnamyl glycoside, rosin, rosavin, rosarin, rosiridin, beta-sitosterol, lotaustralin, picein, or any combination thereof. And another related embodiment provides a pharmaceutical formulation for potentiating chemotherapy in a subject according to any method described above, wherein the pharmaceutical formulation also potentiates radiation therapy.

Still another particular embodiment provides a method for modulating at least one phase of the cell cycle in cancer cells in a subject, the method comprising administering an effective dose of an isolated extract product of Rhodiola crenulata to the subject such that modulation of the at least one phase of the cell cycle in the cancer cells is effected relative to modulation of the at least one phase of the cell cycle in the cancer cells in the absence of administration of the isolated extract product of Rhodiola crenulata. And another related embodiment provides a method for inhibiting cell growth in cancer cells, as indicated by a reduction in S phase of the cell cycle, the method comprising administering an effective dose of an isolated extract product of Rhodiola crenulata to the cancer cells such that the cell growth in the cancer cells is inhibited relative to cell growth in the cancer cells in the absence of administration of the isolated extract product of Rhodiola crenulata, as indicated by a reduction in S phase.

Related embodiments provide a method for modulating at least one phase of the cell cycle cancer cells in, or for inhibiting cell growth in cancer cells as indicated by a reduction in S phase of the cell cycle, further comprising treating the cancer cells with a chemotherapeutic agent before, after, or concomitant with administering the effective dose of the isolated extract product of Rhodiola crenulata.

Another embodiments provides a method for potentiating chemotherapy in a subject being treated with chemotherapy and radiation therapy, the method comprising providing an isolated Rhodiola crenulata extract product comprising a Rhodiola organic extract, aqueous extract, acid extract, neutral extract, essential oil, isolated compound or chemically synthesized equivalent thereof, and administering an effective amount of the isolated Rhodiola crenulata product to the subject being treated with chemotherapy, wherein the subject is being treated with at least one of tamoxifen, a tamoxifen analog, a functional equivalent of tamoxifen, or a salt thereof subjecting the patient to radiation therapy before, after, or concomitant with administering the chemotherapy agent, wherein chemotherapy and radiation therapy treatment in the subject is potentiated relative to such treatment in the absence of the Rhodiola extract product.

Still another embodiment provides a method for potentiating radiation therapy in a subject being treated with radiation therapy, the method comprising providing an isolated Rhodiola crenulata extract product comprising a Rhodiola organic extract, aqueous extract, acid extract, neutral extract, essential oil, isolated compound or chemically synthesized equivalent thereof, and administering an effective amount of the isolated Rhodiola crenulata product to the subject being treated with radiation therapy before, after, or concomitant with administering the radiation therapy, wherein radiation therapy treatment in the subject is potentiated relative to such treatment in the absence of the Rhodiola extract product.

### BRIEF DESCRIPTION OF THE DRAWINGS

The foregoing features of the invention will be more readily understood by reference to the following detailed description, taken with reference to the accompanying drawings, in which:

FIG. 1 is a graph showing an Apoptosis Analysis of 7-AAD Staining in Breast Cancer Cell Lines with 96 hour Rhodiola treatment.

FIG. 2 is a graph showing an Apoptosis Analysis of Annexin & 7-AAD Staining in Breast Cancer Cell Lines Treated with Rhodiola.

FIG. 3 is a graph showing the Effects of Length of Rhodiola Exposure Coupled with Gamma Radiation Treatment in Breast Cancer Cell Lines.

FIG. 4 is a graph showing the Effects of Constant vs. Temporary Exposure to Increasing Levels of Rhodiola in Breast Cancer Cell Lines.

FIG. 5 is a graph showing a Cell Cycle Analysis of 76N Δ239 Cells +/−Rhodiola.

FIG. 6 is a graph showing a Cell Cycle Analysis of 76N Tert Cells +/−Rhodiola.

FIG. 7 is a graph showing an Apoptosis Analysis of 76N Tert Cells +/−Rhodiola, +/−Tamoxifen.

FIG. 8 is a graph showing a Death Time Course of MDA-MB-231 Cells treated with Tamoxifen, Rhodiola, or Tamoxifen plus Rhodiola.

FIG. 9 is a graph showing a NF-KB-luc Reporter Assay on MCF-7 cells in either the Presence or Absence of Rhodiola.

FIG. 10A is a graph showing the in vivo effects of dietary Rhodiola crenulata extracts on survival of mice with a V14 carcinoma graft.

FIG. 10B is a graph showing in vivo effects of different doses of dietary Rhodiola crenulata extracts on survival of BLAB/c mice injected with V14 cells, as compared to controls.

FIG. 11 shows the growth arrest effect of Rhodiola crenulata extracts on V14 cells in vitro over the course of 48 hours.

FIG. 12 shows the apoptosis effect of Rhodiola crenulata extracts on V14 cells in vitro over the course of 72 hours.

FIG. 13 shows the radiation sensitizing effect of Rhodiola crenulata extracts on MDA-MB-231 cells in vitro over the course of 24 hours after 5 Gy of radiation.

FIG. 14 shows Rhodiola-enhanced tamoxifen-induced cell death in vitro on the human ER-negative cell line MDA-MB 231 when pretreated with 10 μM tamoxifen.

FIG. 15 shows the effect of Rhodiola crenulata extract on an in vitro RT-PCR assay of estrogen receptor (ER) levels in MCF-7, T47-D and MDA-MB 231 cells in the presence or absence of 10 μM tamoxifen.

FIG. 16 shows the effect of Rhodiola crenulata extract on telomerase activity after 24 hr in MDA-MB 231 cells in the presence of 10 μM tamoxifen.

FIG. 17 shows the effect of additive/synergistic effect of Rhodiola crenulata extract on telomerase activity after 72 hr in MDA-MB 231 cells in the presence of 10 μM tamoxifen.

FIG. 18 shows in vitro reduction in telomerase activity in various cells lines after treatment with 100 μg/mL Rhodiola crenulata for 24 hrs, as determined using qPCR.

FIG. 19 shows the effect of Rhodiola crenulata extracts on growth arrest of MDA-MB 231 cells given increasing concentrations of all trans retinoic acid (ATRA).

FIG. 20 shows decreased invasion, indicating a decreased ability to metastasize, by MDA-MB 231 cells treated with varying concentrations of Rhodiola crenulata extract, using a matrigel chamber and an in vitro cell wound assay.

FIG. 21 is a Western blot showing in vitro inhibition of AKT phosphorylation in V14 cells treated with 75 μg/mL Rhodiola crenulata over the course of 72 hours using an antibody to phosphorylated-AKT.

FIG. 22 shows in vitro inhibition of cyclooxygenase activity in serum-starved MCF-7, MDA-MB 231, 76N tert and 76N p53 239 cells treated with 100 μg/mL Rhodiola crenulata for 30 minutes.

FIG. 23A shows the apoptotic response in mammary glands from p53 WT or p53 null virgin mice placed in organ culture for 96 hours in the presence or absence of 100 μg/mL Rhodiola crenulata extract, followed by 5 Gy of radiation, as measured by the TUNEL assay (Oncogene—http://www.oncogene.com).

FIG. 23B shows dietary Rhodiola enhances p53 expression in normal mammary glands.

FIG. 23C shows that dietary Rhodiola induces death in a percentage of normal mammary glands.

FIG. 24 is a Western blot showing enhanced phosphorylation of ATM (Ataxia-Telangiectasia Mutated) protein in MCF-7 cells in the presence or absence of overnight treatment with 100 μg/mL Rhodiola crenulata extract, followed by either 0 of 20 Gy radiation exposure using ser 1981, an antibody to phosphorylated ATM.

FIG. 25A shows the activity of certain PLPPP (proline-pentose phosphate pathway) associated enzymes, as a relative percent, in the Rhodiola-sensitive V14a (SCC9) cell line in the presence or absence of 75 μg/mL Rhodiola crenulata extract.

FIG. 25B shows the activity of other PLPPP (proline-linked pentose phosphate pathway) associated enzymes, as a relative percent, in the Rhodiola-sensitive V14a (SCC9) cell line in the presence or absence of 75 μg/mL Rhodiola crenulata extract.

FIG. 26 is a sectionalized graphical representation of the data shown in FIGS. 16A and 16B for the PLPPP associated enzyme activities of interest in the Rhodiola-sensitive V14a (SCC9) cell line, shown as a relative percent.

FIG. 27 is a sectionalized graphical representation of data from a similar study, and for the same PLPPP associated enzyme activities of interest, as those shown in FIGS. 16A and 16B, performed on Rhodiola-resistant V14b cell line, shown as a relative percent.

### DETAILED DESCRIPTION OF SPECIFIC EMBODIMENTS

Definitions. As used in this description and the accompanying claims, the following terms shall have the meanings indicated, unless the context otherwise requires:

As used herein, “extract” or “isolated extract product” means, within the context of the application, a preparation that is in a different form from its source. For example, mechanically lysed cells constitute a cell extract. Other examples include using a solvent to prepare an extract, such as from plant material, soil samples, or animal matter. Possible solvents include water, detergents, oils or organic solvents such as alcohols, ethers, chlorinated hydrocarbons, acetonitrile, esters including ethyl acetate, acidic aqueous solutions, basic aqueous solutions, mixtures thereof. Examples of suitable mixtures include miscible water/alcohol mixtures, or other miscible solvents mixtures that allow appropriate selectivity of the desired polarity of the extracting solvent mixture.

Oils that can be used generally include all oils appropriate for the desired extract. Examples include vegetable oils, including, but not limited to corn, linseed, canola, olive, rapeseed, safflower, sunflower, peanut, walnut, anise, bay, bergamot, castor, cinnamon, clove, coconut, cottonseed, jojoba bean, wheat germ, almond, macadamia, mustard, sesame, flaxseed, and grape seed; animal oils, including but not limited to fish, butterfat from various milk sources, lard; industrially produced, including but not limited to mineral, immersion, and halocarbons. Purified oil components (lipids) may also be used. It is preferable that any and all such combinations may be used, with the guidance that oils and combinations thereof that polymerize or form gums during the extraction procedure may be less preferred (although gum formation may be cured by subsequent addition/extraction with an organic solvent, as appropriate).

Plant extracts of the sort disclosed herein may be made from any part of the Rhodiola plant, from the roots, leaves, stems, bark, from the entire plant, flowers, shoots, cotyledons and seeds, inner cambia, etc. The various parts or whole plant may be dried, or used fresh, frozen, pureed, washed before use, mashed, crushed, ground, or crumbled. Fractionation with additional solvents, such as organic solvents followed by acidic or basic aqueous extraction, may be desired to separate aqueous- and/or acidic- or basic-soluble components and extract products from organic-soluble components and extract products.

Such fractionation procedures can be used to obtain an organic extract (extract product or components soluble in the organic solvent used for the extraction), aqueous extract (extract product or components soluble in the aqueous solvent used for the extraction), acid extract (extract product or components soluble in the acidic solvent used for the extraction), neutral extract (extract product or components that are not soluble in acidic or basic solvents), essential oil, or an isolated compound (an extract product fractionated to a single active component or single active fraction by HPLC or UV analysis, for example).

“Essential oils”, as used herein, refer to distilled (most frequently by steam or water) products obtained from the leaves, stems, flowers, bark, roots, or other elements of a plant and are typically highly concentrated, but may be diluted after preparation, as desired.

“Isolated extract components” as used herein, means an isolated compound, an isolated activity, an isolated group of compounds, an isolated group of activities, and/or any isolated extract component or mixture of components obtained by fractionation from Rhodiola crenulata using solvent or steam extraction processes.

Examples of isolated extract components from Rhodiola crenulata may comprise, but are not limited to, flavonoids (such as quercetin, rutin, and kaempferol), condensed flavonoids (polyphenols, mainly gallic acid and epigallocatechin), cyanoglycosides (which have histamine-inhibiting activity), salidroside, which may constitute approximately 1-2% of the content of concentrated aqueous methanolic extracts of Rhodiola crenulata. Salidroside is comprised of tyrosol linked to glucose; tyrosol is one of the major flavors and aroma ingredients of the olive which confers notable antioxidant activity to olive oil. Rhodiola isolated extract components may also contain sterols, notably daucosterol and sitosterol, and other compounds such as a sachaliside, a flavonoid glycoside, a cinnamyl glycoside, rosin, rosavin, rosarin, rosiridin, beta-sitosterol, tyrosol, lotaustralin, and picein, or combinations thereof. Isolated extract components from Rhodiola crenulata may also comprise other compounds as yet unidentified, but fractionated by assaying for active fractions. In this way, isolated extract components from Rhodiola are obtained comprising compounds exhibiting telomerase inhibition activity, cell growth arrest activity, ER inducing activity, metastases inhibition activity, necrosis inducing activity, p53 inducing activity, or other activities of interest. Assays based on following such activities in various fractions can be used to isolate such active fractions.

For example, an ER alpha IHC staining procedure known to those in the art (see “ER alpha Immunohistochemical Staining Protocol, Copyright© 2003-2005 IHC World, Last modified: Apr. 30, 2005, [online], [retrieved from the internet on May 10, 2005] Retrieved from the IHC World Information Center website<URL: http://www.ihcworld.com/_protocols/antibody_protocols/era_santa_cruz.htm>) can be used to monitor and isolate fractions capable of inducing ER alpha expression in various cell lines of interest, such as breast cancer cell lines. Other assays that could be used to follow active fractions and isolate more concentrated (and pure) active fractions include a TUNEL assay for monitoring apoptosis in cell lines of interest (Guava Technologies—www.guavatechnologies.com/main/products/tunnel-assay.cfm); an MTS assay for indicating chemosensitivity or resistance in cancer cells, for example, to chemotherapy agents with and without Rhodiola extracts (see a description of this assay at Cancer Detection and Prevention Online, International Society of Preventive Oncology,© 1993-2005 by the ISPO; O'Toole, S. A., et al., 2003:27(1), “The MTS assay as an indicator of chemosensitivity/resistance in malignant gynaecological tumours” [online], [retrieved on May 9, 2005]. Retrieved from the Internet URL:http://www.cancerprev.org/Journal/issues/24/101/407/3186; and Cancer Detection and Prevention Online, International Society of Preventive Oncology,© 1993-2005 by the ISPO; O'Toole, S. A., et al., 2000:24 (Supplement1), O'Toole, S. A. et al., “The MTS assay as an indicator of chemosensitivity/resistance in malignant gynaecological tumours” [online], [retrieved on May 9, 2005]. Retrieved from the Internet <URL:http://www.cancerprev.org/Journal/issues/27/1/4778>); or a non-radioactive cell-proliferation assay based on the MTS assay such as available from Promega (Promega CellTiter 96® AQᵤₑₒᵤₛ Non-Radioactive Cell Proliferation Assay (Cat.#G5421)—http://www.promega.com/enotes/applications/0004/ap0017.htm) for monitoring in vitro cytotoxicity of various compounds, such as the effect of chemotherapeutic agents used in concert with Rhodiola extracts.

One of skill in the art could readily identify numerous assays that are useful for monitoring active fractions, and purchase them or have them designed, including a telomerase activity detection kit based on qPCR, such as is available from Allied Biotech, Inc.(http://www.alliedbiotechinc.com/index.asp and described at http://www.alliedbiotechinc.com/ppdfiles/QTD.pdf), or assays to detect DNA damage and/or repair, assays to monitor DNA replication, topoisomerase inhibition assays, and any other readily available or easily designed assay that will allow monitoring, and fractionation, of the activity of interest.

As used herein, “chemically synthesized equivalent” means that the structure of the isolated component has been prepared by means other than an extraction procedure—whether a) by a combination of biosynthesis from bacterial cultures or enzymatic processes (for example) and chemical synthesis to achieve the final isolated or active component, or b) by a total chemical synthesis starting from a defined starting compound or intermediate followed by chemical manipulations and reactions in the absence of enzymes or bacterial or other cultures, or c) by using a bioreactor or other system to produce the isolated or active component biosynthetically with enzymes or cell cultures.

The isolated extract products and extract components can be incorporated into pharmaceutical formulations comprising the extract product, optionally a second therapeutic agent with complementary or synergistic activity for the desired use, and a pharmaceutically acceptable carrier. As used herein, a “pharmaceutically acceptable carrier” means any solvent, dispersion media, coating, antibacterial and antifungal agent, compounds that may delay absorption, fillers, flavors, sweeteners, and diluents such as water, ethanol, saline, sugar solutions, dextrose, human serum albumin, and oils.

An “effective dose,” or a “therapeutically effective dose,” or “effective amount,” as used herein, means any dosage unit form or amount determined to be the amount sufficient to produce the desired effect on the condition, disease, or process. Essential oils, being highly concentrated, will require less extract product to produce the desired effect, typically on the order of only 100 to 300 mg per day or less total dose, whereas more dilute or attenuated extract products may require mg/kg of body weight formulations bringing the total effective dose into the many hundreds of milligrams or more total dose per day. On the other hand, formulations may desire lower amounts to be delivered daily for long-term effects. In such scenarios, the effective dose may be desirable to be 5 to 10 times less than a single daily dose to be delivered only short-term. Highly fractionated components, exhibiting a very high specific activity, or extract products fractionated to a single activity or component, will require the smallest quantity of extract product to achieve an effective, or therapeutically effective dose, often in the micromolar range or less.

The route of administration will also affect the determination and ultimate range chosen for an effective or therapeutically effective dose. For essential oils, it is assumed that oral delivery will result in full absorption of the dose. For topical preparations, if left unoccluded, conservatively less than 50% of the applied dose is expected to be absorbed. Thus, for an application of 10 mL of a 2.5% essential oil concentration, the absorbed dose would be less than 100 mg. Vaginal delivery of a typical 3 g pessary comprising a 10% essential oil concentration would represent a total dose of 300 mg if full absorption is achieved.

Similarly, for rectal suppositories a typical suppository (1 to 3 g) would deliver 250 mg or less if the same 10% essential oil is contained within the suppository, assuming full absorption. Administration by inhalation is about half as efficient. Thus an aerosol diffuser that dispenses 1.0 mL of a 10% essential oil per hour, for a typical adult inhalation session of 15 minutes, with less than 40% inhaled and absorbed, equates to a dose of about 125 mg or less.

In general, appropriate dosage levels will typically be about 0.01 to 10 mg/kg body weight per day, administered as single (where appropriate) or multiple doses over the course of the day. Preferable dosages may be from about 0.01 to about 1 mg/kg/day. Alternatively, dosages may be from about 0.01 to about 0.1 mg/kg/day. Other suitable dose ranges may be as low as 0.001 mg/kg/day, or as high as about 50 mg/kg/day or more.

Oral administration may conveniently be a palatable liquid, or a tablet. Topical applications may be formulated as about 0.0001% to 5% compositions, to be applied 1 to multiple times per day, as appropriate or desired.

Injectable formulations include sterile aqueous and saline solutions, or dispersions and powders to be dissolved/dispersed just prior to administration. Suitable carriers for this type of formulation include physiological saline, bacteriostatic water, phosphate buffered saline, and the like. Sterile solutions are required, and may be achieved through filtration or autoclaving.

“Potentiation” means, in the context of this application to make potent or powerful, to enhance or increase the effect of (a drug), to promote or strengthen (a biochemical or physiological action or effect), or to increase the effect of or act synergistically with (a drug or a physiological or biochemical phenomenon); for example, to “potentiate the drug”. The term includes the concepts of enhancing, heightening, and raising a response or effect, either to a treatment, such as radiation, or a drug, such as a chemotherapeutic drug.

“Analog” means, in the context of this application, a compound related in its core structure that functions equivalently to the parent compound, having minor structural changes in substituents, including modifications to functional groups including hydroxyl, epoxide, ether, carbonyl, ester, acid, amine, amide, thiol, imidazole, thioimidazole, alkene, alkane or aromatic rings, lactones, lactams, to standard derivatives from such functional groups known in the art, including alkoxy groups, esters, acetals, ketals, amides, thioethers, diols, ring-opened or ring-expanded structures, reduced or oxidized functional groups, secondary, tertiary and quaternary amine salts, hydrochloride salts, acetyl groups, etc. “Functional equivalent” means a compound which may different in apparent structure from a comparative compound, but which functions the same as the comparative compound. For example, for tamoxifen, analog means any modification at the dimethylaminoethylether group, the ethyl chain, or replacements of the aromatic benzyl groups which result in a similar planar structure having structure-activity groups important for activity, thus yielding a functionally equivalent tamoxifen analog. Diethylstilbestrol is such an example.

Similarly, for paclitaxel, studies have identified the regions known to be essential for biological activity. In a study conducted at the National Cancer Institute, a C-2 derivative of paclitaxol (Taxol) was more active in promoting tubulin assembly than the original structure. The analogs of paclitaxol found to have increased activity were those with an m-azido group on the benzoyl residue at the C-2 carbon and those with nonreactive meta groups (methoxy and chloro) at the same position (Kowalski, R. J., Giannakokov, P., Hamel, E., J. Biol. Chem., (1997), 272, 2534-2541). Using the knowledge from that study a comparison of a region of absolute stereochemistry shared by both paclitaxol and epothilone was done which determined that fifteen ring atoms and most of the side chain atoms in epothilone can be superimposed onto corresponding atoms in taxol, all with the proper stereochemistry (Winkler, J. D., Axelson, P. H., Bioorganic & Medicinal Chemistry Letters, (1996), 6, 2963-2966), confirming that epothilones are functional equivalents of paclitaxol.

In embodiments of the present invention, it was surprisingly found that Rhodiola crenulata extracts unexpectedly sensitized tumor cells to the effects of radiation, increased the rate of cell death and apoptosis, and induced growth arrest in various tumor cell lines, in contrast to reports with other Rhodiola species showing that extracts and isolated components provide protection against radiation (see, for example “TIBETAN HERBAL MEDICINE With examples of treating lung diseases using Rhodiola and hippophae” by Subhuti Dharnananda, Ph.D., Director, Institute for Traditional Medicine, Portland, Oreg. [online], [retrieved on May 6, 2005]. Retrieved from the Internet URL:<http://www.itmonline.org/arts/tibherbs.htm>). Treatment of mutant or null p53 cell lines with extracts from Rhodiola crenulata surprisingly induced apoptosis, decreased telomerase activity, which may be contributing to the induced apoptosis observed, and arrested cell growth in most immortal and transformed cancer lines examined.

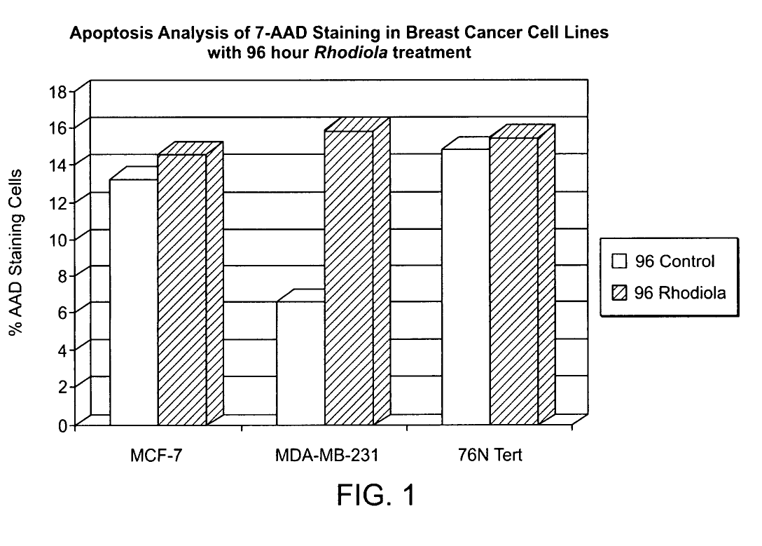

Preliminary results with Rhodiola crenulata are seen in FIGS. 1-9. FIG. 1 shows an apoptosis analysis of 7-AAD staining in breast cancer cell lines with 96 hour Rhodiola treatment. As can be seen, all three breast cancer cell lines examined showed increasing apoptosis after treatment with Rhodiola crenulata compared to the control, with cell lines MCF-7 and Tert-76N showing a modest increase in apoptosis, and cell line MDA-MB-231 showing a substantial increase in apoptosis after treatment.

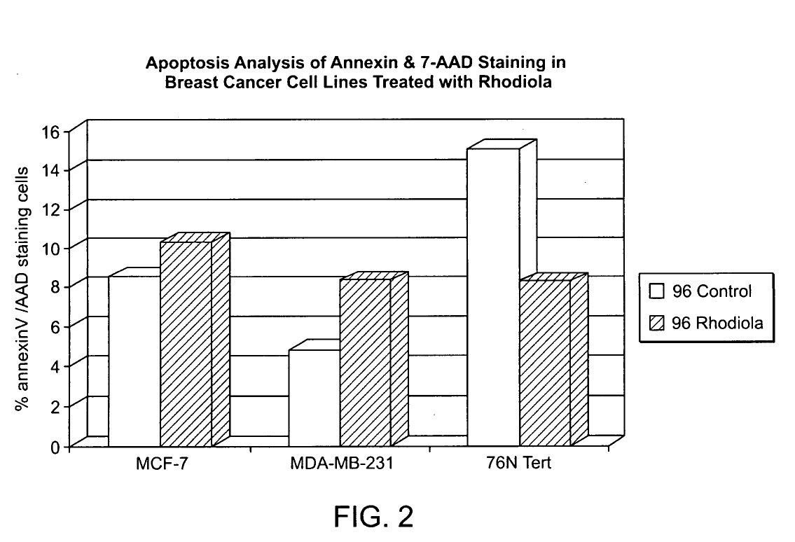

FIG. 2 shows similar results in an apoptosis analysis of annexin & 7-AAD staining in breast cancer cell lines treated with Rhodiola, however in this analysis, the Tert-76N cells did not show an increase in apoptosis compared to the control.

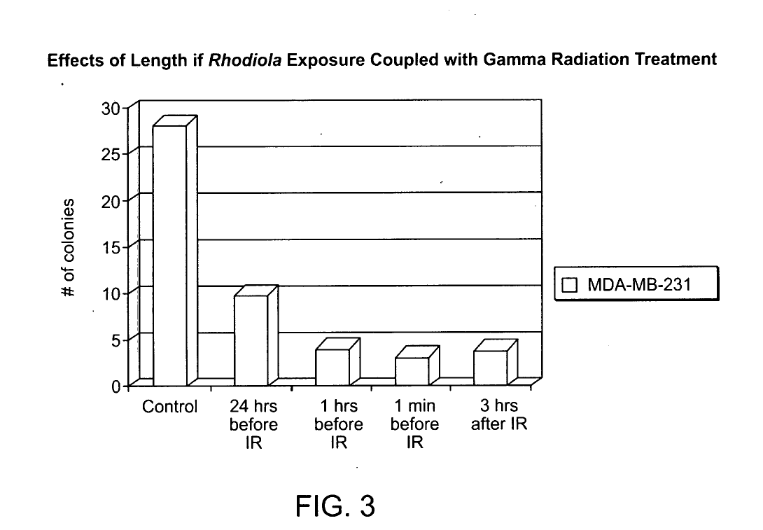

An examination of the effects of time of Rhodiola exposure coupled with gamma radiation treatment in breast cancer cell line MDA-MB-231, as seen in FIG. 3, showed that treatment up to 24 hours before gamma irradiation, or up to 3 hours after irradiation dramatically decreases the number of colonies of cells surviving the treatment. This shows that Rhodiola treatments can potentiate the effect of gamma radiation on cancer cells.

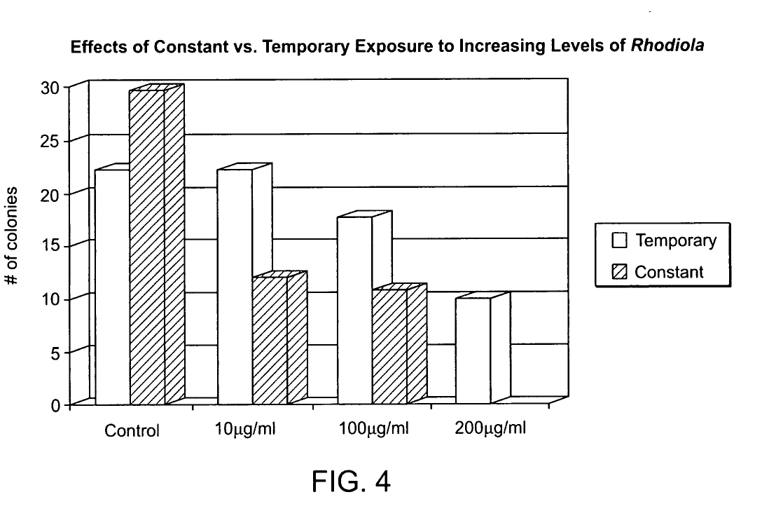

FIG. 4 examined the effects of constant vs. temporary exposure to increasing levels of Rhodiola in breast cancer cell lines. In all dosages, constant exposure reduced colony survival significantly more than temporary exposure to Rhodiola, although temporary exposure at 100 and 200 μg/mL, reduced colony survival compared to the controls.

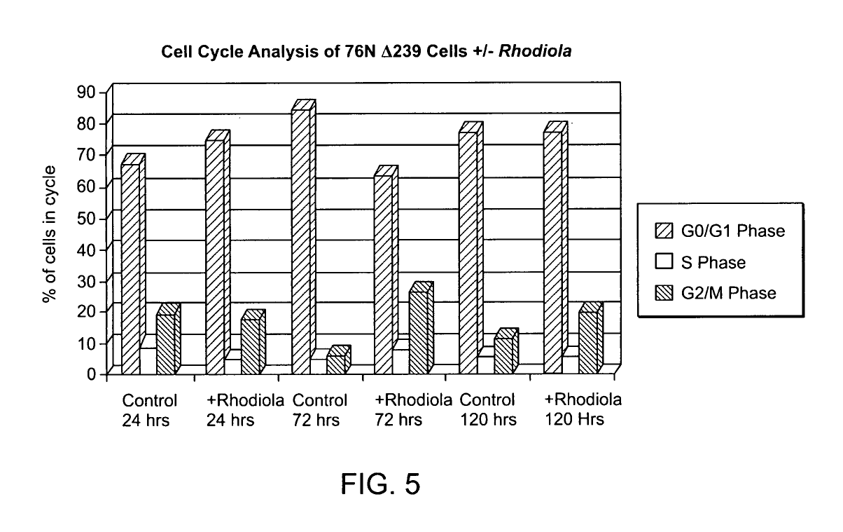

FIG. 5 shows the results of a cell cycle analysis of 76N Δ239 cells in the presence or absence of Rhodiola crenulata. As can be seen, at 24 hours, Rhodiola increases the number of cells in the G0/G1 phase and decreases the number of cells in the S phase, compared to the control. There is little change in the number of cells in the G2/M phase at 24 hours. At 72 hours, Rhodiola decreases the number of cells in the G0/G1 phase, and increases the number of cells in the G2/M phase. There is little change in the number of cells in the S phase at 72 hours. Similar to what was seen at 72 hours, at 120 hours Rhodiola slightly decreases the number of cells in the G0/G1 phase and slightly increases the number of cells in the G2/M phase, but again, there is little change in the number of cells in the S phase.

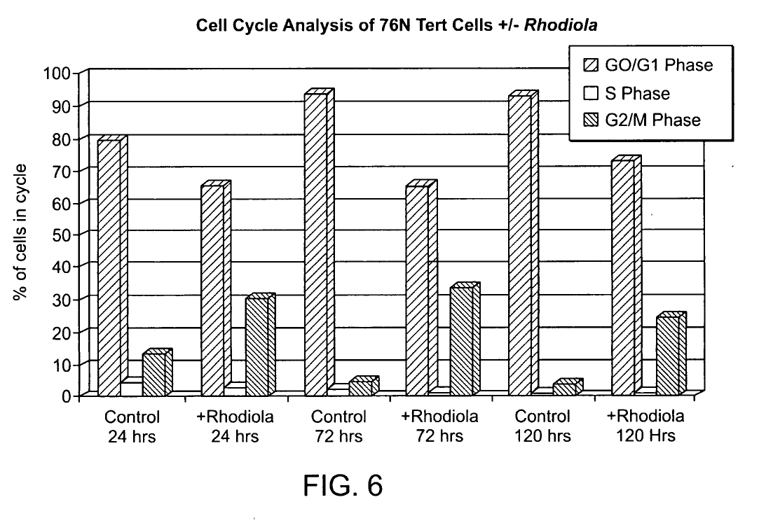

FIG. 6 shows the results of a cell cycle analysis of 76N Tert cells in the presence or absence of Rhodiola crenulata. As can be seen, at 24 hours, Rhodiola somewhat decreases the number of cells in the G0/G1 phase and decreases the number of cells in the S phase, compared to the control. In addition, Rhodiola significantly increases the number of cells in the G2/M phase at 24 hours. At 72 hours, Rhodiola decreases the number of cells in the G0/G1phase, and significantly increases the number of cells in the G2/M phase. There is little change in the number of cells in the S phase at 72 hours. Similar to what was seen at 72 hours, at 120 hours Rhodiola decreases the number of cells in the G0/G1 phase and significantly increases the number of cells in the G2/M phase, but again, there is little change in the number of cells in the S phase. Thus, the effect of Rhodiola effects appear to manifest, at least in part, by a reduction in S phase, although cells will accumulate at G0/G1 or G2/M depending on the p53 status, apparently. A possible reason for the changes in Rhodiola effect observed at later time points may be related to the instability of Rhodiola in culture media, not to any particular effect on cell cycle.

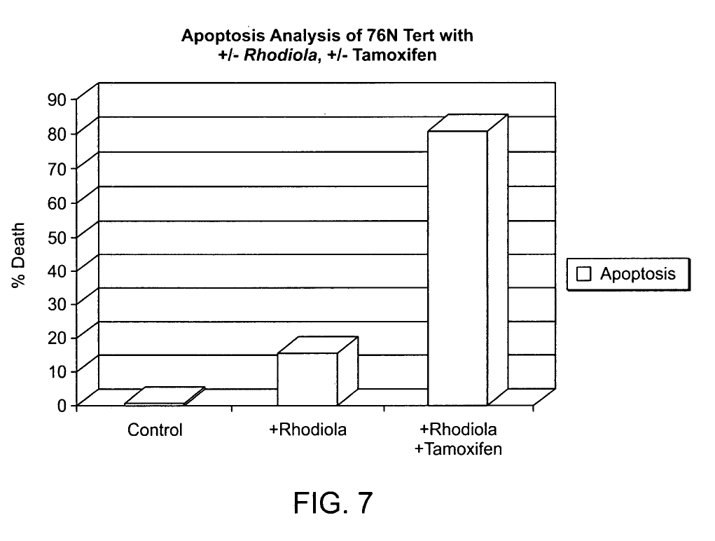

FIG. 7 shows the percentage of cell death for 76N Tert cells exposed to either Rhodiola alone, or Rhodiola and Tamoxifen combined. Rhodiola alone increases the percentage of cell death to about 14%, but Rhodiola plus Tamoxifen increases the percentage of cell death to greater than 80%.

FIG. 8 is similar to FIG. 7, but this experiment involved a time course of MDA-MB-231 Cells treated with Tamoxifen or Rhodiola alone, or Tamoxifen and Rhodiola together. For Rhodiola treatment alone, the greatest increase in cell death was observed at 24 hours, which effect was greater even than Tamoxifen alone at 24 hours. However, at 48 hours, Rhodiola appeared to have no effect on cell death, or to possibly even protect cells from death. In contrast Tamoxifen alone increased the percentage of cell death at both 24 and 48 hours, but the results at neither time were as good as was seen with Rhodiola alone at 24 hours. Finally, Rhodiola combined with Tamoxifen increased the percentage of cell death greater than either Rhodiola alone, or Tamoxifen alone, even at 24 and 48 hours, although, as for Rhodiola alone, the greatest effect was seen at 24 hours.

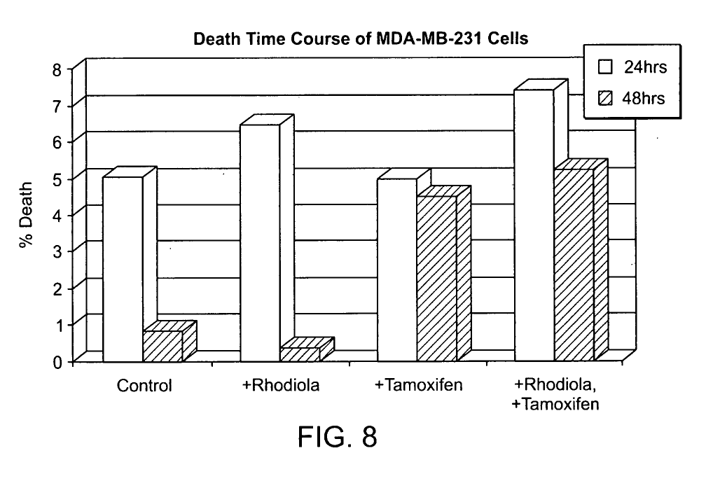

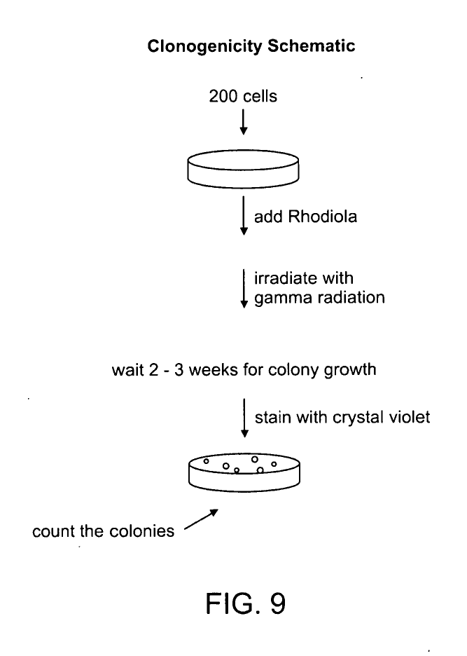

FIG. 9 shows, schematically, how breast cancer cell lines are treated with Rhodiola plus gamma radiation, and then examined for clonogenecity to determine the effects of Rhodiola on radiation therapy in vitro.

### EXAMPLES

### Example 1

### In vivo Effects of Dietary Rhodiola on Survival of V14 Carcinoma Mice

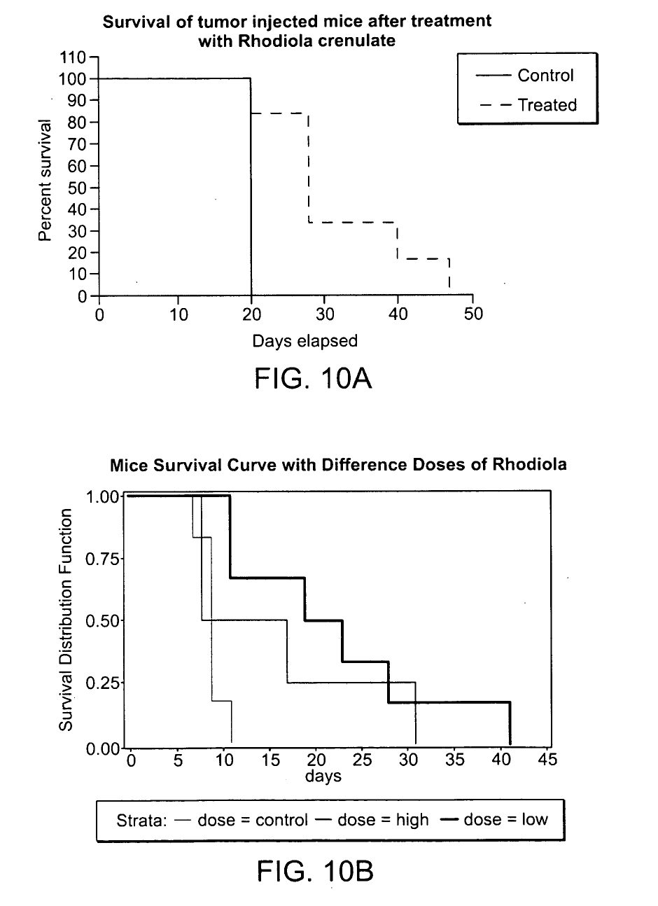

In vivo, dietary Rhodiola crenulata alone decreases the growth and enhances necrosis of an aggressive metastatic breast cancer cell line—V14. V14 cells have mutant p53 and are estrogen receptor-negative (ER(−)), and ErbB2-positive (Her-2(+)). They take on a spindle cell morphology in vivo reminiscent of cells which have undergone an epithelial to mesenchymal transition. Cancers of this type in humans are highly aggressive and resistant to many types of therapies. BALB/c mice were injected subcutaneously in the flank with V14 cells. After the tumor grew to ˜2-3 mm³ in size, the animals were administered water with or without 20 mg/kg/day Rhodiola crenulata extract and treated accordingly for the duration of the experiment. The mice were euthanized when the tumor reached 1 cm³. Subcutaneous injection of V14 cells into the flank caused growth of a tumor by approximately 10 days. The treatment significantly decreased the growth of the tumor and enhanced necrosis, as can be seen in FIG. 10A.

FIG. 10B is a graph showing in vivo effects of different doses of dietary Rhodiola crenulata extracts on survival of BLAB/c mice injected with V14 cells, as compared to controls. Eight-week old BALB/c mice were injected with 5×10⁵ V14 cells and the tumors were allowed to grow to 1-2 mm³. The mice were then put on sham treated water, 0.5 mg/kg/day Rhodiola in water, or 20 mg/kg/day Rhodiola in water. Tumors were measured every 3-4 days and the mice were sacrificed when the tumors reach 1 cm³. Typically, the V14 cell line injected into an immunocompetent mouse develops tumors that reach 1 cm³ within a month. However, mice given 20 mg/kg/day or 0.5 mg/kg/day Rhodiola in their dietary water exhibited tumors with slower growth and higher levels of necrotic tumor death. As can be seen in FIG. 10B, the lower concentration of Rhodiola was just as efficient at slowing growth of the tumors as the higher concentration. Rhodiola extracts lose activity in water after 48 hrs, so the Rhodiola-treated water in these experiments was made fresh daily.

### Example 2

### In vitro Growth Arrest of V14 Carcinoma Cells Treated With Rhodiola

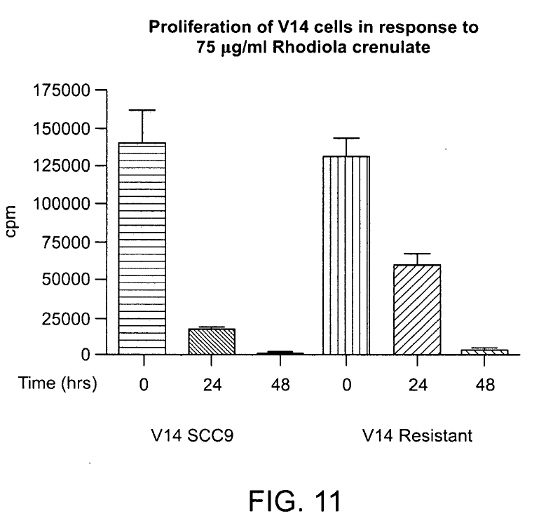

As can be seen in FIG. 11, V14 cells treated with Rhodiola crenulata extract exhibited both anti-growth (i.e. growth arrest) and apoptotic effects over time in vitro. V14 cells were treated with 75 μg/mL Rhodiola and examined for their proliferative potential by ³H thymidine incorporation assays (or apoptotic potential by flow cytometry) at various time points. A greater anti-growth effect was noted in lines which were more sensitive to Rhodiola at lower doses, while the death looked similar. Since there were significant differences in the antioxidant enzymes and energy pathways between the two lines, this suggests that the oxidative state and energy production impacted the growth more than the death phenotype.

### Example 3

### In vitro Apoptosis Study of V14 Carcinoma Cells Treated With Rhodiola

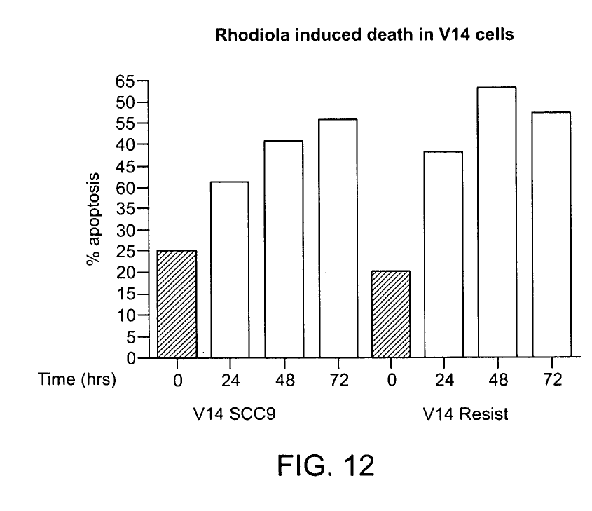

A dose of 75 μg/mL of Rhodiola extract was administered to V14 cells and the amount of apoptosis measured over time by staining with 7-Amino-Actinomycin D (7-AAD) which selectively stains dead cells, followed by flow cytometry analysis. As seen in FIG. 12, Rhodiola treatment induced programmed cell death in both V14 SCC9 cells (single cell clone 9) and V14 resistant cells (a cell line that has some resistance to certain Rhodiola effects) throughout the time course of the experiment, from 24 to 72 hours, although at 72 hours the level of apoptosis began to decrease somewhat in the V14 resistant cells. Hatched bars indicate untreated samples taken from an experiment performed on a different day from that involving treatment with Rhodiola extract.

### Example 4

### Rhodiola-Enhanced Effects of Radiation on MDA-MB-231 Cells

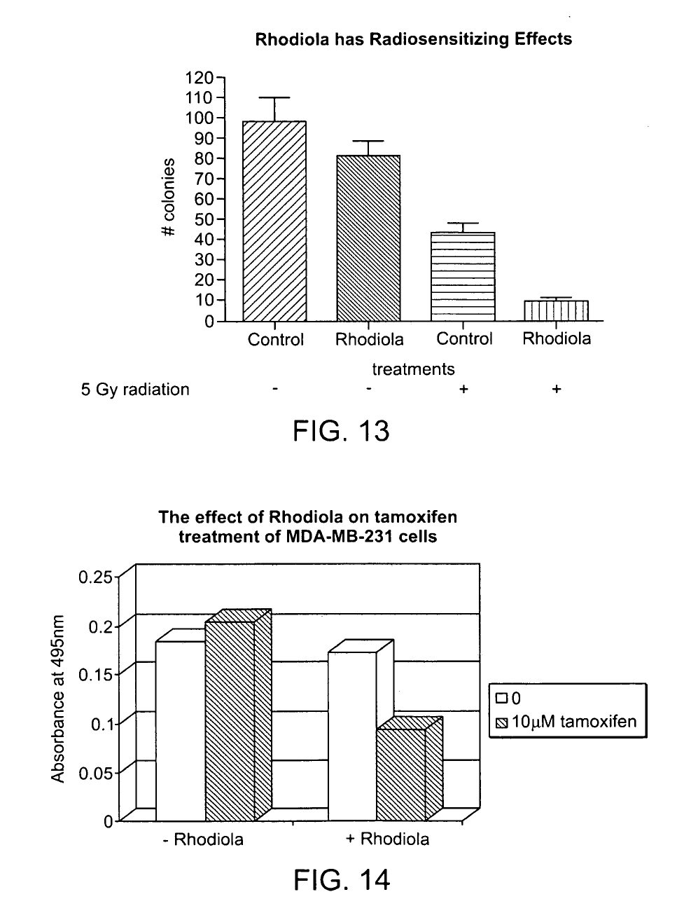

Rhodiola crenulata enhanced the effects of radiation in a clonogenecity assay in the aggressive human metastatic breast cancer line MDA-MB-231. 100 MDA-MB-231 cells were plated and exposed to Rhodiola extract at 100 μg/mL for 24 hours prior to exposure to 5Gy of radiation. After 2-3 weeks of growth which allowed clones to form the cells were fixed and stained with crystal violent and the colonies were then counted. The colonies were smaller and fewer after Rhodiola/radiation treatment, suggesting that both death and proliferation were a factor in the response observed. FIG. 13 shows the number of colonies (Y-axis) after treatment with Rhodiola crenulata extract alone (checkerboard), radiation alone (horizontal lines), or radiation plus Rhodiola crenulata treatment (vertical lines).

### Example 5

### Rhodiola-Enhanced Effects of Tamoxifen-Induced Death in hER(−) and (+) Cells

As seen in FIG. 14, pre-treatment of MDA-MB-231 cells with 100 μg/mL Rhodiola crenulata for 24 hours followed by treatment with 10 mM tamoxifen resulted in significantly increased cell death, as indicated by a decrease in absorbance at 495 nm (Y-axis) as monitored by the MTS assay which measures conversion of the MTS tetrazolium compound (3-(4,5-dimethylthiazol-2-yl)-5-(3-carboxymethoxyphenyl)-2-(4-sulfophenyl)-2H-tetrazolium, inner salt) by cells to formazan by dehydrogenase enzyme activity. These results indicate that Rhodiola crenulata extracts enhance tamoxifen responsiveness in ER(−) cells such as this breast cancer cell line. Tamoxifen is a selective estrogen receptor modulator, making it excellent as therapy for ER positive tumors, but not for ER negative tumors. Thus, the ability of Rhodiola crenulata to enhance the tamoxifen-induced cell death in ER(−) cells is particularly significant, and represents a novel and exciting opportunity in chemotherapy for particularly aggressive and hard to treat cancers.

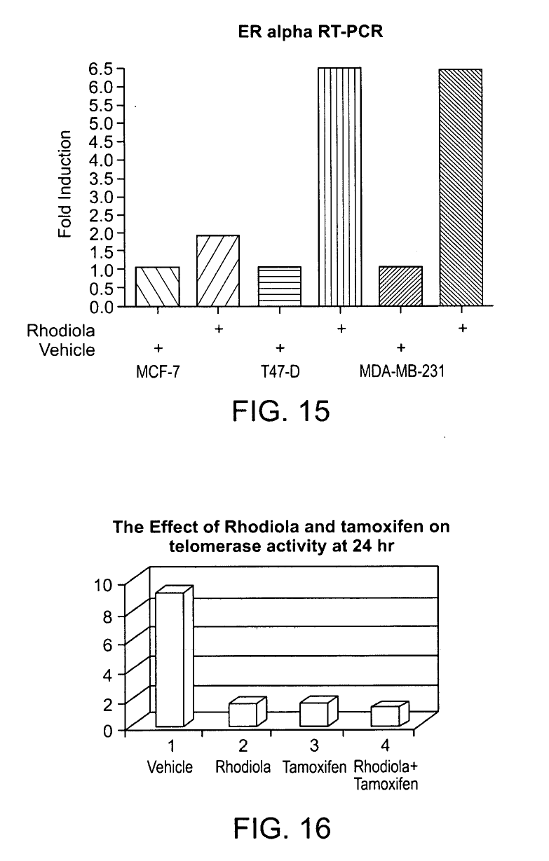

FIG. 15 shows up-regulation of ER alpha in various cell lines, both ER(−) (MDA-MB-231) and ER(+) (MCF-7 and T47-D) by treatment with 100 μ/mL Rhodiola crenulata, as monitored by RT-PCR. As can be seen, Rhodiola doubled the level of ER alpha in MCF-7 cells (checkerboard) and quintupled the levels of ER alpha seen in T47-D (horizontal/vertical lines) and MDA-MB-231 cells (diagonal lines). It is possible that Rhodiola crenulata extracts sensitize ER(−) cells to tamoxifen by modulating the level of estrogen receptors. Cells in culture quickly lose estrogen receptor status, and exogenous expression actually causes the arrest in growth of such cells. In human therapy there are concerns that tamoxifen, which kills off estrogen responsive cells, may be selecting for ER(−) cells. Thus, the present results showing that Rhodiola crenulata can increase the levels of estrogen receptor in ER(+) and ER(−) cells are especially interesting. These results suggest that the observed up-regulation of ER, in particular the ER beta receptor, may be how Rhodiola crenulata sensitizes cells to treatment with tamoxifen.

### Example 6

### Effect of Rhodiola and Tamoxifen on Telomerase Activity in MDA-MB-231 Cells

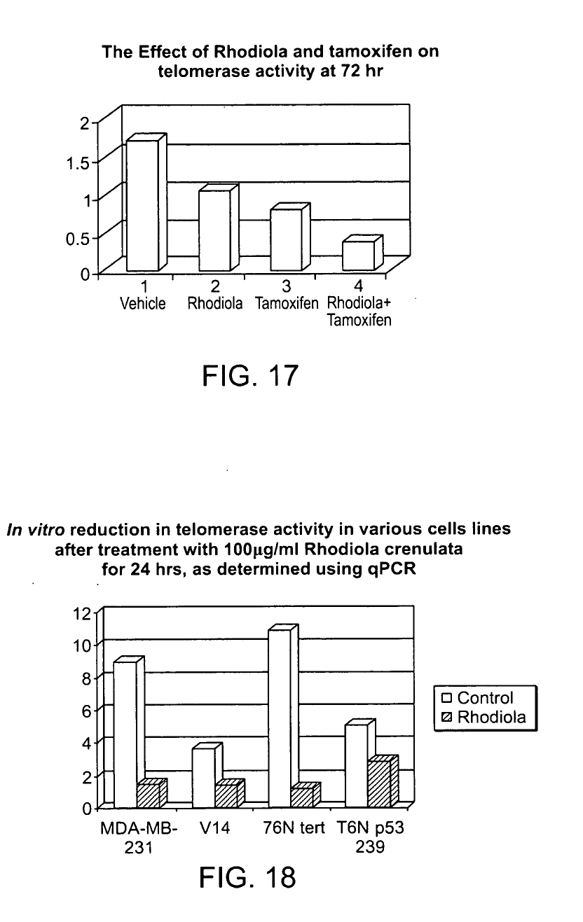

FIGS. 16 and 17 show the effect of Rhodiola crenulata (100 μg/mL) and tamoxifen (10 μM) on telomerase activity in human MDA-MB-231 breast cancer cells at 24 hr and 72 hr, respectively. As can be seen in FIG. 16, at 24 hours both Rhodiola crenulata and tamoxifen inhibited telomerase activity at about the same extent, and little effect was seen when the cells were treated with both. By 72 hours, however, as seen in FIG. 17, telomerase activity is starting to return for cells treated with either Rhodiola extract or tamoxifen alone, but when cells are treated with both, telomerase activity was even more decreased, suggesting that the two agents work by different mechanisms, and are thus more effective when they are used together, resulting in additive or synergistic decreases in telomerase activity.

Additional cell lines were examined for the effect of Rhodiola crenulata enhancement of tamoxifen activity, as seen in FIG. 18. The breast cancer cell lines MDA-MB-231, V14, 76N tert (an immortalized epithelial breast cancer cell line), and 76N p53 239 (immortalized by presence of a dominant negative p53 gene) were treated as indicated above for 24 hours at 100 μg/mL Rhodiola crenulata. As seen, in the presence of Rhodiola (dark bars, labeled “Rhodiola”) all four cell lines showed enhanced decrease in telomerase activity relative to the effects seen with no Rhodiola (light bars, labeled control).

### Example 7

### Effect of Rhodiola on Cell Growth Arrest by Retinoids in MDA-MB-231 Cells

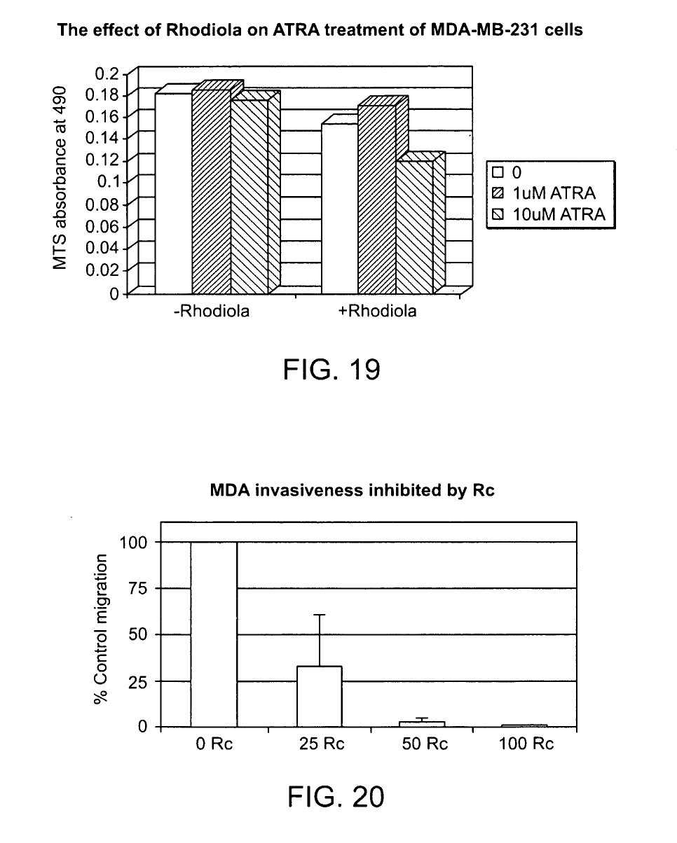

Rhodiola crenulata extracts were administered to MDA-MB-231 cells in the presence or absence of either 1 μM or 10 μM all trans retinoic acid (ATRA), and cell viability/cytotoxicity was monitored using the MTS assay, as described above, and monitored for absorbance at 490 nm as indicative of chemosensitivity/resistance to the ATRA administered. FIGS. 19 shows that in the absence of Rhodiola crenulata, MDA-MB-231 cells showed little difference in chemosensitivity to ATRA (−Rhodiola). In contrast, in the presence of Rhodiola, at 10 μM ATRA plus 100 μg/mL of Rhodiola crenulata for 96 hours, a decrease in absorbance was observed (far right bar, +Rhodiola) indicating increased chemosensitivity in these sells that were so treated. Comparable effects were seen with Rhodiola and other retinoids, including 9-cis retinoic acid and N-(4-hydroxyphenyl) retinamide (HPR), a synthetic derivative of ATRA (data not shown).

### Example 8

### Rhodiola crenulata Effect on Invasiveness by MDA-MB-231 Cells

FIG. 20 shows the results of a matrigel invasion assay performed with human MDA-MB-231 cells treated with increasing concentrations of Rhodiola crenulata, from zero to 100 μg/mL. The extent of migration of the MDA-MB-231 cells through the matrigel material is indicated in the Y-axis, as the percent of migration relative to the control. In this study, MDA-MB-231 cells were serum starved and then placed on top of matrigel in a trans-well chamber. The cells were treated with the concentrations of Rhodiola indicated, and then 1% serum is added back. After ˜24 hours the number of cells to migrate through the matrix was counted. As can be seen, increased concentrations of Rhodiola crenulata extract showed a dramatically decreased extent of migration by the MDA-MB-231 cells such that at 100 μg/mL, almost no migration was observed.

Similar assays were performed with mouse V14 cancer cells with similar results observed. Other studies with mouse V14 and V6 breast cancer cells investigated cell migration in a wound assay. In this assay, V-6 or V14 cells are plated at a density of approximately 7×10⁴ cells/cm² in serum-free medium on laminin-coated plates incubated in serum-free medium for 24 hours. The confluent monolayer is scratched to create a small cell-free wound area. Rhodiola is then added and 1hour later 1% serum is added. After 6 hours the cells which have migrated across the wound are counted. In the wound healing assays, Rhodiola extracts inhibited the ability of the V6and V14 cells to migrate across the wound (as was seen in the matrigel invasion assay) increasing concentrations of Rhodiola resulted in decreased cell migration and lack of wound healing. The mechanisms behind these affects by Rhodiola will provide important insights into effective and therapeutic use of the Rhodiola crenulata extract in chemoprevention and in the treatment of other cancers and diseases.

### Example 9

### Effect of Rhodiola on AKT Phosphorylation and Cyclooxygenase Activities in Breast Cancer Cells

The protection of cancer cells during assault from radiation or chemotherapeutic agents are often due to up-regulated survival pathways, such as those engaged by receptor tyrosine kinases. These receptors often signal through PI3 kinase to AKT, a kinase which is involved in activating a number of other survival proteins. Inhibition of AKT activity has been shown to sensitize many types of tumor cells to radiation and therapeutics. The increase in AKT activity in ER positive breast tumors is believed to be partially responsible for tamoxifen resistance.

Resistance can also be attributed to the increased activity of cyclooxygenase (COX) activity likely through its ability to regulate the PI3 kinase pathway. Rhodiola extracts are able to inhibit both AKT and COX activities in breast cancer cell lines. Cyclooxygenase is an enzyme that increases inflammatory response through the production of prostaglandins. It is also expressed in tumor cells and maintains the proliferative and anti-apoptotic pathways, as well as enhancing angiogenesis. In the past few years, epidemiological studies have indicated that NSAIDS (non-steroidal anti-inflammatory drugs) can act in a chemopreventive fashion for cancers of the colon and possibly other tissues, such as breast tissue. We investigated the effect of Rhodiola crenulata on cyclooxygenase activity and have found that it can reduce COX activity in multiple cells lines.

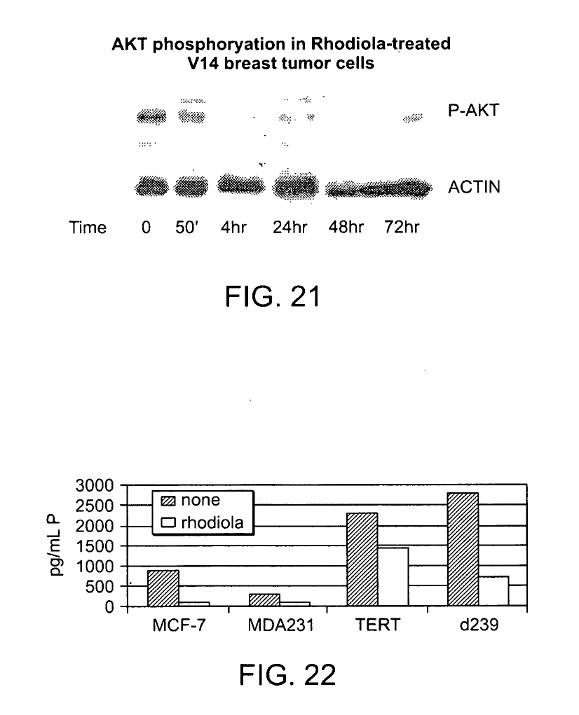

FIG. 21 shows a Western blot of V14 cells treated with 75 μg/mL Rhodiola extract over a period of 72 hours, at the time intervals indicated. Protein lysates were obtained by incubating cells for 30 minutes at 4° C. in a buffer containing 50 mM Tris-HCl (pH 7.4), 10 mM NaCl, 0.5% NP40, 100 mM NaF, 10 mM MgCl₂ and a protease inhibitor cocktail (Sigma, St. Louis, Mo., USA), followed by centrifugation at 12,000 rpm for 20 min. Cell lysates were then subjected to SDS-PAGE (10% polyacrylamide, 1% SDS). After transfer to PVDF membranes, the membranes were blocked and then probed with antibodies against phosphorylated AKT activity (ser 1981). Parallel samples were also probed for B-actin to normalize for loading differences. As can be seen, at increasing times, P-AKT activity decreases until it is almost not observable (see times 4 hr or longer).

FIG. 22 shows inhibition of cyclooxygenase activity by Rhodiola crenulata extracts in a panel of breast cancer cell lines, including MCF-7, MDA-MB-231, 76N tert, and 76N p53-239. As shown, treatment with 100 μg/mL of Rhodiola for thirty minutes with serum-starved cells treated with arachidonic acid (AA) decreased cyclooxygenase activity, as determined by measuring levels of PGE₂ (prostaglandin E₂), expressed in pg/mL, in all cell lines examined, compared to control experiments. Lysates were prepared and PGE₂ was measured using a PGE₂ EIA kit.

Results show almost complete inhibition of cyclooxygenase activity in MCF-7 and MDA-MB-231 cells, as indicated by pg/mL PGE₂ measured (Y-axis) after 30 minutes. Even the immortalized cell lines 76N tert and 76N p53 239 exhibited decreased cyclooxygenase activity after treatment with Rhodiola extract.

### Example 10

### Effect of Rhodiola Extract on Mouse Mammary Gland Sensitivity to Radiation

Previous studies have shown that mouse virgin mammary glands are refractive to gamma radiation. Neither p53 nor p21 are up-regulated in such glands, and death does not occur, suggesting that there is a mechanism in place in the mammary glands that blocks p53 reactivity. We have found that pregnancy, or pregnancy levels of estrogen, progesterone or retinoic acid will release p53 from this refractory state and allow for stabilization and activation of this tumor suppressor (Kuperwasser et al., 2000; Tu and Schneider, unpublished). Thus we examined Rhodiola crenulata extracts for their ability to sensitize the mammary gland to radiation-induced cell death in virgin mice.

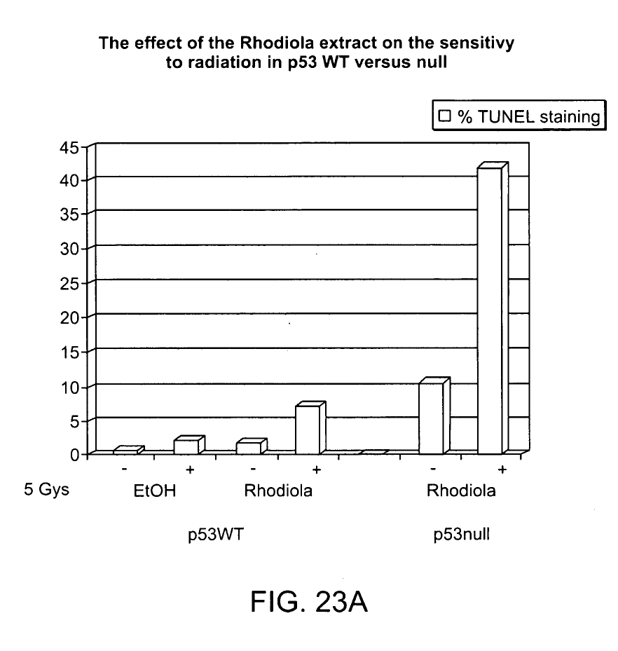

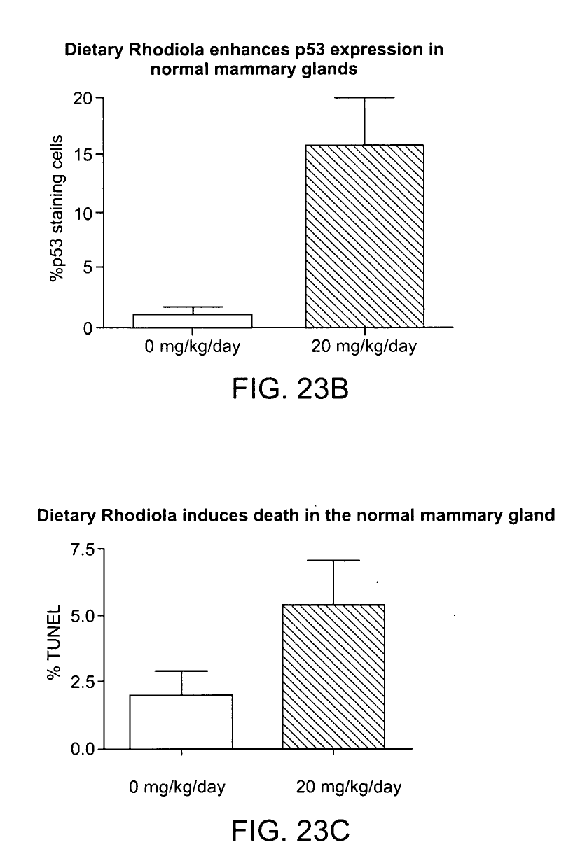

We found that incubation of mammary gland organ cultures from virgin mice with Rhodiola extracts (100 μg/mL final) imparted a sensitization of epithelial cells to death, especially in p53 null tissues (see FIG. 23A). In cell lines, Rhodiola crenulata appears to be able to growth arrest most immortal and transformed cell lines, and also decrease telomerase activity in these lines. However, in our studies so far, an increase in death is preferentially observed in p53 mutant or null cells.

This increase in death may be due to mitotic catastrophe which is observed in p53 mutant lines when telomerase is inhibited (Preto et al., 2004). Telomerase is the enzyme which replicates the telomeres at the ends of chromosomes. In normal cells the enzyme is low and after a set number of cell doublings the chromosomes shorten to a point where the cell undergoes senescence and growth arrests. In cancer cells this enzyme activity is high and gives the cancerous cell a seemingly endless life span. Rhodiola was able to significantly decrease telomerase activity in numerous mammary epithelial cell lines suggesting that it might work by limiting the replicative life span of cancer cells.

In FIG. 23A, mammary glands from p53 WT or p53 null virgin mice were placed in organ culture for 96 hours in the presence or absence of 100 μg/mL Rhodiola crenulata extract. The glands were then subjected to 0 or 5 Gy radiation and incubated for 6 hours to allow for death to occur. The glands were fixed and paraffin embedded. The apoptotic response was measured by TUNEL assay (Oncogene—www.oncogene.com).). The Y-axis represents % death. FIG. 23B shows results from other experiments indicating that dietary Rhodiola extract enhances p53 expression.

Curiously, Rhodiola crenulata also appears to induce death in a percentage of cells from normal mammary glands (see FIG. 23C). Although this phenomenon is not yet understood, given the other data showing that Rhodiola extracts induce apoptosis in mammary gland tumor cells, and increase expression of p53, it is possible that the “normal” cells observed to be susceptible to Rhodiola extract have actually sustained non-lethal mutations such that they have not yet undergone cell death, but making them susceptible to Rhodiola, so that after treatment with Rhodiola, such cells die. It is also possible that these Rhodiola-susceptible “normal” mammary cells represent a particular fraction of cells that just happen to be proliferating rapidly, again making them susceptible to Rhodiola treatment. Or, they may simply represent a fraction of cells that are susceptible for some other as-yet unidentified reason. What is known is virgin mouse mammary gland cannot activate p53 in response to irradiation or induce death in response to irradiation/DNA damage, so the induction of p53 and death suggests a release of these activities and then possibly a response to something else (reactive oxygen intermediates being one possibility).

Rhodiola's effect on p53 mutant and p53 null cancer cell lines suggests that it may be a powerful agent in the assistance of inducing cell death in cancer cells impaired in this tumor suppressor (about 50% of breast cancers). The release of p53 from its inactive state appears to be a common event upon treatment with ovarian hormones and retinoids. Given this data, we suspect that the release of p53 may also be an important marker for determination of strong chemopreventive agents as both ovarian hormones and retinoids can reduce tumor induction by 50%.”

### Example 11

### Rhodiola-Induced Enhancement of Phosphorylated ATM

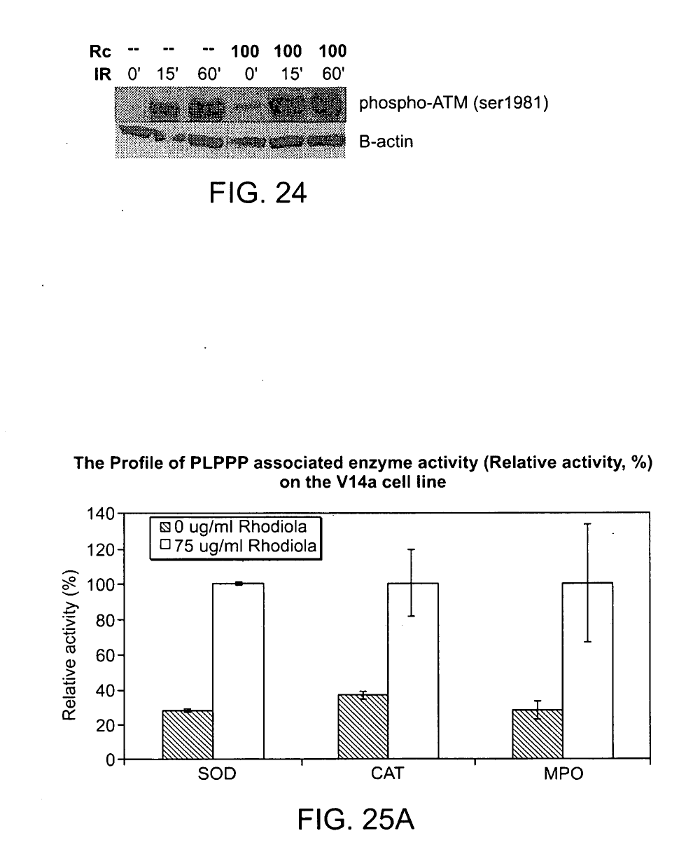

In an attempt to define how this was occurring, we looked at levels of the DNA damage sensing protein Ataxia Telangiectasia Mutated (ATM) and found that Rhodiola extract sensitizes cells to a heightened ATM activation in response to radiation. MCF-7 cells were incubated in the presence or absence of 100 μg/mL Rhodiola crenulata overnight, and were then exposed to either 0 or 20 Gy of ionizing radiation. Cell lysates were collected 15 and 60 minutes after irradiation, as well as from un-irradiated controls (0′, IR). Proteins were separated by SDS-PAGE, transferred to PVDF membrane, and probed for phosphorylated-ATM (phospho-ATM (ser1981). Parallel samples were also probed for B-actin, to normalize for loading differences. As shown in FIG. 24, a Western blot investigating how Rhodiola enhances the phosphorylation (and thus activation) of ATM, increasing radiation doses from 0 to 60 minutes of zero or 20 Gy ionizing radiation resulted in substantial increase in ATM activity.

The increase in ATM activity may be due to a lowering of the radiation threshold by Rhodiola. It has been demonstrated that previous exposure to agents which activate ATM on their own (see control) can cause an exaggerated response to radiation later. This also may suggest that in cancer cells Rhodiola is incapable of mounting a full fledged antioxidant response to the early increase in reactive oxygen species and that this helps to sensitize the cancer cells. In fact, there was an attenuated antioxidant response, but the increase in death, ATM, and p53 suggests that the increase in antioxidant enzymes was not enough to counteract the damaging effects of the reactive oxygen species.

In other types of normal cells we have data suggesting that there will be a strong increase in the antioxidant enzymes in response to Rhodiola and this is through the proline-linked pentose phosphate pathway (PLPPP). In the tumor cells, Rhodiola was unable to push the cells into the proline linked pathway as indicated by the accumulation of proline and the decrease in proline dehydrogenase activity. Other energy pathways were also compromised suggesting that there may have been an energy crisis at the level of oxidative phosphorylation (due to dysfunctional ATP generating steps in the mitochondria) and that this led to the promotion of growth arrest.

### Example 12

### Rhodiola Effect on PLPPP Associated Enzyme Activity

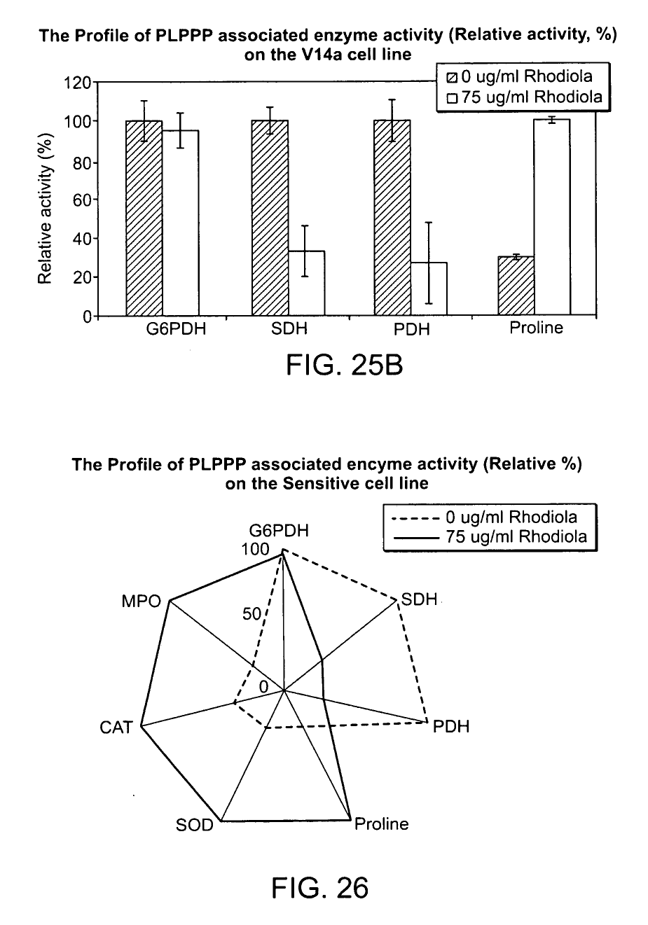

The effect of Rhodiola crenulata extract on the profile of proline-linked pentose phosphate pathway (PLPPP) associated enzymes was investigated. V14 cells were treated with 75 μg/mL Rhodiola crenulata. As seen in FIG. 25A, the antioxidant enzymes superoxide dismutase (SOD), catalase (CAT) and myeloperoxidase (MPO) show increased levels in the presence of Rhodiola (white bars) relative to levels in the absence of Rhodiola (dark bars), but these levels are significantly lower than the levels of these enzymes induced in normal cells by Rhodiola crenulata treatment(data not shown). This indicates that Rhodiola extracts protect regular, non-diseased cells from oxidative cell death, but paradoxically promote cell death in cancer cells. This effect probably results because cancer cells have redox breakdown (that is linked to protective PLPPP and optimum antioxidant enzyme response in normal cells) and so utilize alternative pathways linked to Krebs/Tricarboxylic acid for oxidative phosphorylation for energy. This means that the standard protective PLPPP-linked redox pathways are dysfunctional in cancer cells and therefore results in more oxidative stress. Rhodiola extracts appear to force cancer cells to revert to using the standard redox pathways, but since these pathways are dysfunctional in cancer cells, the cancer cells die due to energetically unsustainable excessive oxidative stress as indicated by the SOD, CAT and MPO data.

In support of this, after 48 hours, cells treated with 75 μg/mL Rhodiola show a shift in enzyme activities required for metabolic pathways, with an accumulation of proline indicating that these tumor cells are unable to utilize the proline-linked pentose phosphate pathway. FIG. 25B shows no significant change in levels of glucose-6-phosphate dehydrogenase (G6PDH) but a large increase in the level of proline, and decreased levels of succinate dehydrogenase (SDH) and proline dehydrogenase (PDH) in the presence of Rhodiola (white bars) relative to levels in the absence of Rhodiola (dark bars). The accumulation of proline corresponds to lack of activity of PDH and therefore dysfunctional PLPPP-linked redox pathways that are protective in normal cells.

FIG. 26 shows the same information as that present in FIGS. 25A and 25B with solid lines representing enzyme levels in the presence of 75 μg/mL Rhodiola and dashed lines representing enzyme levels in the absence of Rhodiola for the Rhodiola-sensitive cell line SCC9.

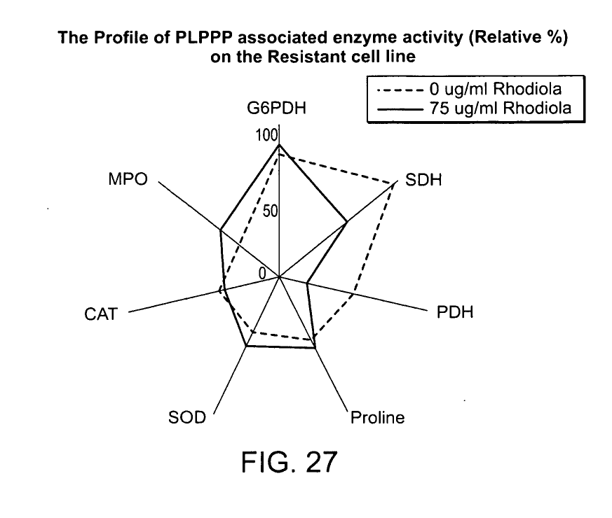

FIG. 27 shows similar information for Rhodiola-resistant cell line V14b (not a true resistant line, but so characterized because of certain results seen under some conditions—data not shown). As indicated, in this cell line, little differences are seen between G6PDH, MPO, CAT, SOD and proline levels in the presence (solid lines) or absence (dotted lines) of Rhodiola, although SDH and PDH levels are decreased in cells treated with 75 μg/mL Rhodiola (see solid lines). The normal G6PDH levels means that activity of SOD, CAT and MPO are supported by NADPH of the pentose phosphate pathway as excessive oxidative pressure sets in, with poor utilization of proline and low PDH activity in response to Rhodiola in V14 cell line.

While the current data relates primarily to breast cancer cell lines, other types of tumors are encompassed by embodiments of the invention using Rhodiola crenulata in conjunction with radiation therapy or chemotherapy for improved response and treatment. In particular, up-regulation of AKT, telomerase activity and Cox-2 activity are hallmark features for numerous cancers. The ability of Rhodiola crenulata extracts to inhibit cyclooxygenase activity, alter metabolic pathways, increase antioxidant enzymes also indicates its usefulness and applicability as a chemopreventive agent to decrease the risk of cancer in many tissues.

Purification and isolation of extracts from Rhodiola crenulata follow standard procedures known in the art. The assays used to follow activity include three high throughput assays to identify the important constituents of Rhodiola. These assays test 1) for the ability of the fraction to inhibit migration (scratch-wound assay), 2) for the ability to cause apoptosis under conditions of stress (propidium iodide staining) and 3) for the ability to alter mitochondrial energy pathways (MTS assay). The bulk of the activity has been identified to reside in the hydrophilic fraction. In addition, the apoptosis activity appears to involve multiple components, indicating that a combination of isolated components and/or isolated extracts may be beneficial for potentiating chemotherapy, in addition to administration of isolated components and isolated fractions alone. Moreover, the apoptosis active components do not bind to carbon columns and bind only weakly to cation exchange columns.

Results indicate that Rhodiola crenulata extracts (Barrington Nutritionals, Harrison, N.Y.) sensitized certain breast cancer cell lines, immortalized epithelial cells, and a subset of ductal cells in mammy glands to the effects of various treatments, such as radiation, retinoids, and anti-cancer compounds such as tamoxifen, cyclophosphamide and others. With regards to the ability of Rhodiola to compliment other therapeutic strategies, we have shown that Rhodiola extracts work with tamoxifen to keep levels of telomerase down for longer periods of time in ER(−) cell lines, and further to augment the growth inhibition seen in cancer cells by tamoxifen and retinoids. Experimental data also indicates that Rhodiola crenulata extract inhibits the growth of cells, inhibits cyclooxygenase (COX) activity and telomerase activity, and enhances cell death in certain tumor cells. Evidence also suggests that Rhodiola crenulata extracts appear to act in a strongly chemopreventive manner, particularly in breast cancers and colon cancers, and similar results are expected in ovarian, uterine, leukemia.

What is claimed is:

1. A method for potentiating chemotherapy in a subject being treated with chemotherapy, the method comprising:
providing an isolated Rhodiola crenulata extract product comprising a Rhodiola organic extract, aqueous extract, acid extract, neutral extract, essential oil, isolated compound or chemically synthesized equivalent thereof; and
administering an effective amount of the isolated Rhodiola crenulata product to the subject being treated with chemotherapy, wherein chemotherapy treatment in the subject is potentiated relative to such treatment in the absence of the Rhodiola extract product.

2. A method for potentiating chemotherapy in a subject according to claim 1, wherein the subject is being treated with at least one of tamoxifen, paclitaxel, docetaxel, epothilone, a retinoid, progesterone, cyclophosphamide, methotrexate, bleomycin, cisplatin, oxaliplatin, carboplatin, nitrogen mustards, melphalan, mechlorethamine, dacarbazine, lomustine, carmustine, chlorambucil, vinca alkaloids including vincristine and vinblastine, topotecan, ironotecan, etoposide, doxorubicin, idarubicin, epirubicin, daunorubicin, mitoxantrone and related molecules, gemcitabine, 5-fluorouracil, mitomycin C, any functional equivalent thereof, or any combination thereof.

3. A method for potentiating chemotherapy in a subject according to claim 2, wherein the subject also receives radiation therapy.

4. A method for potentiating chemotherapy in a subject being treated with chemotherapy and radiation therapy, the method comprising:
providing an isolated Rhodiola crenulata extract product comprising a Rhodiola organic extract, aqueous extract, acid extract, neutral extract, essential oil, isolated compound or chemically synthesized equivalent thereof; and
administering an effective amount of the isolated Rhodiola crenulata product to the subject before, after, or concomitant with being treated with chemotherapy and radiation therapy, wherein the subject is being treated with at least one of tamoxifen, paclitaxel, docetaxel, epothilone, a retinoid, progesterone, cyclophosphamide, methotrexate, bleomycin, cisplatin, oxaliplatin, carboplatin, nitrogen mustards, melphalan, mechlorethamine, dacarbazine, lomustine, carmustine, chlorambucil, vinca alkaloids including vincristine and vinblastine, topotecan, ironotecan, etoposide, doxorubicin, idarubicin, epirubicin, daunorubicin, mitoxantrone and related molecules, gemcitabine, 5-fluorouracil, mitomycin C, any functional equivalent thereof, or any combination thereof;
wherein chemotherapy and radiation therapy treatment in the subject is potentiated relative to such treatment in the absence of the Rhodiola extract product.

5. A method for potentiating chemotherapy in a subject according to claim 3 or 4, wherein the subject is being treated with a chemotherapy agent comprising tamoxifen, paclitaxel, docetaxel, epothilone, a functional equivalent thereof, an analog thereof or a salt thereof.

6. A method for potentiating chemotherapy in a subject according to claim 3 or 4, wherein the subject is being treated with a retinoid.

7. A method for potentiating chemotherapy in a subject according to claim 5, wherein the subject is being treated with a chemotherapy agent comprising tamoxifen, a tamoxifen analog, a functional equivalent of tamoxifen, or a salt thereof.

8. A method for potentiating chemotherapy in a subject according to claim 2 wherein the subject being treated with a chemotherapy agent comprising tamoxifen, paclitaxel, docetaxel, epothilone, a functional equivalent thereof, an analog thereof or a salt thereof.

9. A method for potentiating chemotherapy in a subject according to claim 2, wherein the subject is being treated with a retinoid.

10. A method for potentiating chemotherapy in a subject according to claim 8, wherein the subject is being treated with a chemotherapy agent comprising tamoxifen, a tamoxifen analog, or a salt thereof.

11. A method for promoting cell death in cancer cells the method comprising administering an isolated extract product of Rhodiola crenulata to the cancer cells such that the cancer cells show increased cell death relative to cancer cells in the absence of the isolated extract product of Rhodiola crenulata.

12. A method for enhancing the growth arrest effect of a retinoid on cancer cells, the method comprising administering an isolated extract product of Rhodiola crenulata to the cancer cells in the presence of a retinoid such that the cancer cells show enhanced growth arrest relative to cancer cells in the absence of the isolated extract product of Rhodiola crenulata.

13. A method for inhibiting the progression of metastases in cancer cells, the method comprising administering an isolated extract product of Rhodiola crenulata to the cancer cells such that the cancer cells show inhibited progression of metastases relative to cancer cells in the absence of the isolated extract product of Rhodiola crenulata.

14. A method for decreasing telomerase activity in cancer cells, the method comprising administering an effective dose of an isolated extract product of Rhodiola crenulata to the cancer cells such that the cancer cells show decreased telomerase activity relative to cancer cells in the absence of the isolated extract product of Rhodiola crenulata.

15. A method for decreasing telomerase activity in cancer cells according to claim 14, further comprising administering an effective dose of tamoxifen to the cells, wherein the cancer cells are ER-negative breast cancer cells.

16. A method for decreasing cyclooxygenase activity in cancer cells, the method comprising administering an effective dose of an isolated extract product of Rhodiola crenulata to the cancer cells such that the cancer cells show decreased cyclooxygenase activity relative to cancer cells in the absence of the isolated extract product of Rhodiola crenulata.

17. A method for modulating the activity of p53 in cancer cells, the method comprising administering an effective dose of an isolated extract product of Rhodiola crenulata to the cancer cells such that the cancer cells show modulated p53 activity relative to cancer cells in the absence of the isolated extract product of Rhodiola crenulata.

18. A method for increasing ataxia telangiectasia mutated (ATM) activity in cancer cells, the method comprising administering an effective dose of an isolated extract product of Rhodiola crenulata to the cancer cells such that the cancer cells show increased ATM activity relative to cancer cells in the absence of the isolated extract product of Rhodiola crenulata.

19. A method for inhibiting utilization of the proline-linked pentose phosphate pathway in cancer cells, as indicated by at least one of an accumulation of proline, a decreased level of succinate dehydrogenase, or a decreased level of proline dehydrogenase, the method comprising:
administering an effective dose of an isolated extract product of Rhodiola crenulata to the cancer cells such that the utilization of the proline-linked pentose phosphate pathway in cancer cells is inhibited relative to utilization of said pathway in cancer cells in the absence of administration of the isolated extract product of Rhodiola crenulata.

20. A method for effecting cell death of cancer cells in a subject with cancer, the method comprising administering an effective dose of an isolated extract product of Rhodiola crenulata to the subject such that cell death of the cancer cells is effected relative to cell death of the cancer cells in the absence of administration of the isolated extract product of Rhodiola crenulata.

21. A method for modulating AKT phosphorylation activity in estrogen receptor-positive or estrogen receptor-negative breast cancer cells, the method comprising administering an isolated extract product of Rhodiola crenulata to the ER-positive or ER-negative breast cancer cells such that the breast cancer cells show modulated AKT phosphorylation activity relative to breast cancer cells in the absence of the isolated extract product of Rhodiola crenulata.

22. A method according to any of claims 14-20, wherein the cancer cells are breast, colon, ovarian, liver, head and neck, lung, pancreas, leukemia, melanoma, and uterine cancer cells or any combination thereof.

23. A pharmaceutical formulation for potentiating chemotherapy in a subject, the formulation comprising:
a therapeutically effective amount of an isolated Rhodiola extract product isolated from the species Rhodiola crenulata or a chemically synthesized equivalent thereof;
a therapeutically effective amount of a chemotherapeutic agent; and
a carrier.

24. A pharmaceutical formulation for potentiating chemotherapy in a subject according to claim 23, wherein the isolated Rhodiola extract product comprises an organic extract, aqueous extract, miscible aqueous/organic mixture extract, acid extract, base extract, neutral extract, an essential oil, or a combination thereof.

25. A pharmaceutical formulation for potentiating chemotherapy in a subject according to claim 24, wherein the isolated extract product comprises an aqueous extract.

26. A pharmaceutical formulation for potentiating chemotherapy in a subject according to claim 24, wherein the isolated extract product comprises an organic extract.

27. A pharmaceutical formulation for potentiating chemotherapy in a subject according to claim 24, wherein the isolated extract product comprises a neutral extract.

28. A pharmaceutical formulation for potentiating chemotherapy in a subject according to claim 24, wherein the isolated extract product comprises a miscible aqueous/organic mixture extract.

29. A pharmaceutical formulation for potentiating chemotherapy in a subject according to claim 24, wherein the isolated extract product comprises an aqueous methanol extract.

30. A pharmaceutical formulation for potentiating chemotherapy in a subject according to claim 24, wherein the isolated extract product comprises an essential oil.

31. A pharmaceutical formulation for potentiating chemotherapy in a subject according to claim 24, wherein the chemotherapy agent comprises at least one of tamoxifen, paclitaxel, docetaxel and related molecules, a retinoid, progesterone, cyclophosphamide, methotrexate, bleomycin, cisplatin, oxaliplatin, carboplatin, nitrogen mustards, melphalan, mechlorethamine, dacarbazine, lomustine, carmustine, chlorambucil, vinca alkaloids including vincristine and vinblastine, topotecan, ironotecan, etoposide, doxorubicin, idarubicin, epirubicin, daunorubicin, mitoxantrone and related molecules, gemcitabine, 5-fluorouracil, mitomycin C, any functional equivalent thereof, or any combination thereof.

32. A pharmaceutical formulation for potentiating chemotherapy in a subject, wherein the Rhodiola product is an isolated extract that comprises a flavonoid, a condensed flavonoid, a cyanoglycoside, a salidroside, tyrosol, a sterol, a sachaliside, a flavonoid glycoside, a cinnamyl glycoside, rosin, rosavin, rosarin, rosiridin, beta-sitosterol, lotaustralin, picein, or any combination thereof.

33. A pharmaceutical formulation for potentiating chemotherapy in a subject according to any of claims 23-32, wherein the pharmaceutical formulation also potentiates radiation therapy.

34. A method for modulating at least one phase of the cell cycle in cancer cells in a subject, the method comprising:
administering an effective dose of an isolated extract product of Rhodiola crenulata to the subject such that modulation of the at least one phase of the cell cycle in the cancer cells is effected relative to modulation of the at least one phase of the cell cycle in the cancer cells in the absence of administration of the isolated extract product of Rhodiola crenulata.

35. A method for inhibiting cell growth in cancer cells, as indicated by a reduction in S phase of the cell cycle, the method comprising:
administering an effective dose of an isolated extract product of Rhodiola crenulata to the cancer cells such that the cell growth in the cancer cells is inhibited relative to cell growth in the cancer cells in the absence of administration of the isolated extract product of Rhodiola crenulata, as indicated by a reduction in S phase.

36. A method according to claim 34 or 35, further comprising treating the cancer cells with a chemotherapeutic agent before, after, or concomitant with administering the effective dose of the isolated extract product of Rhodiola crenulata.

37. A method for potentiating chemotherapy in a subject being treated with chemotherapy and radiation therapy, the method comprising:
providing an isolated Rhodiola crenulata extract product comprising a Rhodiola organic extract, aqueous extract, acid extract, neutral extract, essential oil, isolated compound or chemically synthesized equivalent thereof; and
administering an effective amount of the isolated Rhodiola crenulata product to the subject being treated with chemotherapy, wherein the subject is being treated with at least one of tamoxifen, a tamoxifen analog, a functional equivalent of tamoxifen, or a salt thereof;
subjecting the patient to radiation therapy before, after, or concomitant with administering the chemotherapy agent;
wherein chemotherapy and radiation therapy treatment in the subject is potentiated relative to such treatment in the absence of the Rhodiola extract product.

38. A method for potentiating radiation therapy in a subject being treated with radiation therapy, the method comprising:
providing an isolated Rhodiola crenulata extract product comprising a Rhodiola organic extract, aqueous extract, acid extract, neutral extract, essential oil, isolated compound or chemically synthesized equivalent thereof; and
administering an effective amount of the isolated Rhodiola crenulata product to the subject being treated with radiation therapy before, after, or concomitant with administering the radiation therapy;
wherein radiation therapy treatment in the subject is potentiated relative to such treatment in the absence of the Rhodiola extract product.
